SEQUENCE LISTING (38 sequences)

SEQ ID NO: 1 - DNA, 2573 bp, Arabidopsis thaliana
atggagcttgaagaacagaagaagtactttcatggaacactagagattacaatcttcgatgcaacacctttttctcctccatttcctttcaatgtaagtctatatcatatactgaattagtgactctgatgatgttatcattgtgttcattttcatgagcttttgttatctttgttcacagtgtatatgtacaaagccaaaagcagcttacgtgaccatcaagataaacaagaagaaagttgctaaaacaagctcagaatacgaccgtatttggaatcagacttttcagatcctctgcgctcacccggttactgacacgactatcaccatcacgctcaagactcgatgttctgttctcggaagattccgaatctctgcagaacagatcctgacatctaattcagcagttatcaacgggttcttccctctgattgcagacaatgggtcaacgaaacgtaacttgaagcttaagtgtttgatgtggttcagaccggcttatcttgaaccgggatggtgcagagcgcttgaagaagcgtcttttcaaggtattaggaacgcaagcttccctcaaagatcgaactgcagagtcgtactgtatcaagatgcacaccacaaggccacatttgatccaagggttgatgatgttccttttaatgcaaggaatctatgggaagatgtttacaaagcgatcgaaagtgctaggcacttggtttacattgcaggatgggcactaaaccctaatcttgttcttgtacgcgacaacgaaactgagattccacatgcggttggagtaacggttggcgagcttctgaaacggaaatcagaggaaggtgtagcggtgagagtaatgttgtggaacgacgagacgtctttaccaatgatcaagaacaaaggtgtaatgagaacaaacgtcgaaagagctcttgcttacttcagaaacacaaacgttgtttgtagattgtgtccaagattgcacaaaaagctccctacagcgtttgcacaccaccaaaagacaataacactggatacacgggtgacaaattcaagcacgaaggagcgagagatcatgagcttccttggcgggtttgacctttgcgatggtcgatatgacactgaggagcattcactgtttcgtacactcggaacagaagctgatttttaccagacaagtgtagctggagctaagctaagtagaggcggaccaagagagccatggcatgattgccatgtgtccgtggttggaggagcggcttgggatgtcctgaagaacttcgaacagcgatggacgaaacagtgtaacccttccgtgttggttaacacttcagggatcagaaacttagtaaacctaaccggacctactgaggaaaataacaggaaatggaatgttcaagttctgagatcgatagatcacatatcggcaacagagatgccaagaggtttgccggtagagaagagtgtacacgatggctatgtcgcagcaattcgaaaggctgagaggtttatctacatcgagaatcagtatttcatgggaagctgtgatcattgggagagcaagaacgataagtacgaaatttctcttaacgaagctattaaatatccattgggagattctgatctattttagtatttctttttttgatggatagaatctgcagcggatgcacaaacttgatacctgtcgaaatagcgttaaagattgctgctaagatacgtgcaagggagagatttgctgtgtatatagtcataccgatgtggccggaagggccaccggagagtgaaacggttgaagagatacttcattggacgagagagacgatgtcaatgatgtatcagatcattggagaggcgatatgggaagttggagacaagtcgcatccgagggattatctaaacttcttctgtcttgcaaacagagaagagaagagagatggggagtttgaagcagtttcgtcgccccaccagaaaacgcattactggaatgcgcagaggaatcggagattcatggtctatgtccactccaagctcatgataggtaacaaacaatccgcaattcacaatctgaaatgaaaatatattgaagattcgttaaactttcacaaactttcacttaagatttaaattacagagattgtcatatgtttgtagtggacgacacatacattttgataggatcagccaatataaaccagagatcaatggatggttgtcgggacaccgagattgcaataggttgctaccaaaccaataccaacaacaccaacgaaatccaagcttaccggttatcgctctggtatgagcataccggtggcaagatcacggcagatgacttatcatcgtcagagccagagagtcttgaatgtgtccgagggttacggacaataggagagcaaatgtgggagatttacagtggagacaaggtcgtagacatgctcggtatccatttggtcgcctatccaattagtgtgacaggagatggtgcagttgaggaagtcggtgacggttgctttcctgataccaagaccttggtgaaaggaaagaggtccaagatgtttcctcctgttctaaccacctga

SEQ ID NO: 2 - DNA, 3308 bp, Arabidopsis thaliana
atgacggagcaattgctgcttcatggaactcttgaagtgaagatttataggatcgataagttgcatcaacgttcaagattcaatttatgcggcaaggtaatatttaatttcaacactatctactaataagagatattagacgaaggttacttgtaaaataagactcaaaaatgattcataggggaacaaggagccaacaggtaaaaagactcaatctcaaatcaaaagactaacggactcatgcacaagtttgtttggaggacatctatacgcaactattgacctagacaggtcaagagtggctagaaccatgatgagacgtcatcctaaatggttacaatccttccacgtctacaccgcacattcaatctctaaaatcatattcacagttaaagaagatgaaccggtcagtgctagtttgatagggcgagcttatttacctgtaacagaagtcatcaccggacaacctatagaccgatggctcgatatcttagacgagaacaggagaccaatccaaggaggttccaaactccatgtacgtgtgaagttcactcatgtgacacaggacgtgaattggaacaaaggaataatattaccttccttcaatggagttcctaacgcttatttcaaccaaagagaaggttgcaaagttacactataccaagacgctcacgttcttaacgaataccctgatgttaccctcacgggaggacaagttatatacaagcatcatcggtgttgggaagagattttcgatgcgatatgggaagcaaaacatttgatatacatcgctggttggtccgtaaacaccgacgtgacattagttagagatcctaaacggacgagaccgggaggggacctcaagttaggggaattgctgaagaagaaagcagaagaaaacgtgaccgtgctgatgcttgtgtgggacgatagaacgtctcacgaagtttttaagagagacggtttgatgatgactcatgatcaagaaacttacgattacttcaagaacacgaaagtgcgttgcgttctctgtccgcggaatccggataatggtgatagcattgtgcaagggttcgaggtcgctactatgtttactcatcatcaaaaaacgattgtggtggacagtgaagtggacgggtcattaaccaagagaaggatagtgagttttcttggaggtattgatctttgtgatgggagatacgacactgtagagcatcctttgtttggtactttgaatagtgttcatgctaatgactttcaccagccgaactttgatggtgcatcgattaaaaagggcggtccacgtgaaccgtggcacgatattcactgcaagctggatggtcctgcggcgtgggatgttttatacaattttgagcagaggtggatgaaacaaggtattgaaacaaattcacaatgcttcaacgttttctttttgcgtttacatgttacaataattggtgattttttcgaatacaaattttttttaatcgtgaaaatgttaacgaaccaagtgtcgttagctctattggtaaagaccgaccctaagcgagacttagaaatcaccagttcgagtgacgcttgggacgaaactaatattgtaatttgtgttttcgggtctaaagggattaagaattttttttgtttgaattacgggcttttgcattaacattattaaaacacacataaatttgtgtgtcaaacaacggtagccctaaacaaaagtgaaatgtcattctagaaactcacagaattcaatcattaattcgttagaaatttattttatgtttttaggtagtggtagaaggtaccttatatcgatggcacagctcgcagagatcacggtcccgccacttccgattgtacaaccagacaatgaagaaggttggacagttcaagtcttccggtcgatagatgatggtgcagttgaagggttcccagaggatccacgtgaagctgcaagcataggacttataagcggaaaagataacgtgatcgaaagaagcattcaagacgcttacgttaatgccataaggagagccaaaaacttcatctacattgagaaccaatattttctaggaagctcctttggatggaactctagagacataaatttaaatgagatcaatgcactacaactcattcctaaagagatttctctaaaaatcgtgagtaaaatcgaggcaggcgagcggttttcggtgtatatagtgattccgttatggcctgaaggtaaacctggttccgcatcggttcaagcaatactagactggcaaaggagaacaatggagatgatgtatactgatataatcattgcactacgtaaaaaaggtttagatgcaaaccctagagactacctaacgtttttctgcctcgggaaccgggaaaagggcaaggtgggtgaatacttgcctccggaaaaaccggaggctaattctgattacgcaagagctcaagaatctcgtcggttcatgatctatgtccactctaaaatgatgattggtaagtccaaatttacctttcataactcatcatttttatttggctttaaggaatgtggcgtttataagagacaacaactaggattagtggtgggtcatgtcgggctgaagaccgaatgagtctagttaatctcgttaggcccattttttccgaatatagtgttaaattataataaaagataaaaaaagtagcggttttacaataagtgcatcgtttgtttgataaaaaaatttaaacgtaaaaagtttataaaaatttaacttactgatttaaaccaaaatttttaaattttgtttggccaattgctacaaacgtattaagaacaatcaaaaagctcttaacatggttcatatatctatgcagttgatgatgagtacataataataggatcagccaatataaaccaaaggtcaatggatggaggtcgagacacagagatcgccatgggagcatatcaacctagccatctcctatctactaataatatgaggccggttggccaaatatttagcttcaggatatcactatggcttgaacacctacgagtaacaaccaatgcatttcagtgccccgaaagcgaagagtgtataagaatggttaatgcaacggcagacgagctatggggactatactcagcacaagagtatcctcgaaatgacgatttaccggggcatttgcttagttatccgattagcattggtagcaatggggaagtaacaaatcttgctgggactgaattctttcctgatactaacgcaaaggttgtaggcgaaaaatctaattatcttcctccaatccttacttcttag

SEQ ID NO: 3 - DNA, 33 bp, Artificial sequence
  PLDepsilon 45 forward primer
agagggatccatggagcttgaagaacagaagaa

SEQ ID NO: 4 - DNA, 32 bp, Artificial sequence
  PLDepsilon 43 reverse primer
gttaggcctggtggttagaacaggaggaaaca

SEQ ID NO: 5 - DNA, 22 bp, Artificial sequence
  T-DNA left border primer
gcgtggaccgcttgctgcaact

SEQ ID NO: 6 - DNA, 24 bp, Artificial sequence
  PLDepsilon primer forward
tatcttgaaccgggatggtgcaga

SEQ ID NO: 7 - DNA, 24 bp, Artificial sequence
  PLDepsilon primer reverse
tagggtttagtgcccatcctgcaa

SEQ ID NO: 8 - DNA, 30 bp, Artificial sequence
  PLDepsilon complementation primer forward
gaattcgcgtcctcgcatgtctcaggtaaa

SEQ ID NO: 9 - DNA, 33 bp, Artificial sequence
  PLDepsilon complementation primer reverse
ggatcctgccctcatgtgttcttatcaaggaca

SEQ ID NO: 10 - DNA, 32 bp, Artificial sequence
  PLDalpha3-specific primer forward
ctcgagatgacggaccaattgctgcttcatcg

SEQ ID NO: 11 - DNA, 31 bp, Artificial sequence
  PLDalpha3-specific primer reverse
acgcctagaagtaaggatgattggaggaaga

SEQ ID NO: 12 - DNA, 22 bp, Artificial sequence
  PLDalpha3 left border primer
gcgtggaccgcttgctgcaact

SEQ ID NO: 13 - DNA, 24 bp, Artificial sequence
  PLDalpha3 null mutant primer forward
atggttaatgcaacggcagacgag

SEQ ID NO: 14 - DNA, 24 bp, Artificial sequence
  PLDalpha3 null mutant primer reverse
cccggtaaatcgtcatttcgagga

SEQ ID NO: 15 - DNA, 33 bp, Artificial sequence
  PLDalpha3 complementation primer forward
ctgcaggtaagattcacaaaattggtgtaatac

SEQ ID NO: 16 - DNA, 30 bp, Artificial sequence
  PLDalpha3 complementation primer reverse
aagcttgagtgaatatggtctatggatatt

SEQ ID NO: 17 - DNA, 27 bp, Artificial sequence
  TeasyAsc5 primer
atggcgcgccatatggtcgacctgcag

SEQ ID NO: 18 - DNA, 25 bp, Artificial sequence
  TeasyAsc3 primer
atggcgcgcccgacgtcgcatgctc

SEQ ID NO: 19 - DNA, 32 bp, Artificial sequence; identical to SEQ ID NO: 10
  PLDalpha3 gene specific primer forward

SEQ ID NO: 20 - DNA, 31 bp, Artificial sequence; identical to SEQ ID NO: 11
  PLDalpha3 gene specific primer reverse

SEQ ID NO: 21 - DNA, 24 bp, Artificial sequence
  PLDalpha3 forward primer
atggttaatgcaacggcagacgag

SEQ ID NO: 22 - DNA, 24 bp, Artificial sequence
  PLDalpha3 reverse primer
cccggtaaatcgtcatttcgagga

SEQ ID NO: 23 - DNA, 22 bp, Artificial sequence
  RD29B forward primer
acaatcacttggcaccaccgtt

SEQ ID NO: 24 - DNA, 24 bp, Artificial sequence
  RD29B reverse primer
aactcacttccaccggaatccgaa

SEQ ID NO: 25 - DNA, 24 bp, Artificial sequence
  RAB18 forward primer
gcagtcgcattcggtcgttgtatt

SEQ ID NO: 26 - DNA, 24 bp, Artificial sequence
  RAB18 reverse primer
acaacacacatcgcaggacgtaca

SEQ ID NO: 27 - DNA, 24 bp, Artificial sequence
  TOR forward primer
agttcgaagggcaaagtacgacga

SEQ ID NO: 28 - DNA, 24 bp, Artificial sequence
  TOR reverse primer
tacgcacgctcatagctctccaaa

SEQ ID NO: 29 - DNA, 24 bp, Artificial sequence
  AGC2.1 forward primer
agaaacgtctcttccgcttcacca

SEQ ID NO: 30 - DNA, 24 bp, Artificial sequence
  AGC2.1 reverse primer
acctgaagaatctgacacggccaa

SEQ ID NO: 31 - DNA, 24 bp, Artificial sequence
  FT forward primer
tccctgctacaactggaacaacct

SEQ ID NO: 32 - DNA, 24 bp, Artificial sequence
  FT reverse primer
acgatgaattcctgcagtgggact

SEQ ID NO: 33 - DNA, 24 bp, Artificial sequence
  BFT forward primer
attcaaacagagagggaggcaagc

SEQ ID NO: 34 - DNA, 24 bp, Artificial sequence
  BFT reverse primer
gcagcaacaggttgagaaagacca

SEQ ID NO: 35 - DNA, 24 bp, Artificial sequence
  TSF forward primer
aagacaaacggtttatgcaccggg

SEQ ID NO: 36 - DNA, 24 bp, Artificial sequence
  TSF reverse primer
ttgaagtaagaggcagccacagga

SEQ ID NO: 37 - DNA, 22 bp, Artificial sequence
  UBQ10 forward primer
cacactccacttggtcttgcgt

SEQ ID NO: 38 - DNA, 24 bp, Artificial sequence
  UBQ10 reverse primer
tggtctttccggtgagagtcttca

(19) United States
(12) Patent Application Publication
(10) Pub. No.: US 2010/0037351 A1
(43) Pub. Date: Feb. 11, 2010

(54) Alteration of Phospholipase De (PLDe) or Phospholipase Da3 (PLD a3) Expression in Plants

(75) Inventors: Xuemin Wang, St. Louis, MO (US); Yueyun Hong, St. Louis, MO (US)

Correspondence Address: SENNIGER POWERS LLP, 100 NORTH BROADWAY, 17TH FLOOR, ST LOUIS, MO, 63102, US

(73) Assignees: DONALD DANFORTH PLANT SCIENCE CENTER, St. Louis, MO (US); THE CURATORS OF THE UNIVERSITY OF MISSOURI, Columbia, MO (US)

(21) Appl. No.: 12/412,992

(22) Filed: Mar. 27, 2009

Related U.S. Application Data
(60) Provisional application No. 61/040,508, filed on Mar. 28, 2008.

(51) Int. Cl. A01H 5/00; C12N 15/82

(52) U.S. Cl. 800/290; 800/298; 800/278

(57) ABSTRACT

Transgenic plants and seeds with altered expression of phospholipase Dε (PLDε) are described, i.e., plants and seeds either overexpressing or underexpressing PLDε. Methods for producing such transgenic plants and for increasing a plant's ability to capture and utilize nitrogen, increasing a plant's biomass production, increasing the yield of a plant or increasing a plant's ability to grow under hyperosmotic stress by overexpressing PLDε are also disclosed. Further disclosed are transgenic plants and seeds which overexpress phospholipase D alpha3 (PLDα3), methods for producing such transgenic plants and seeds, and methods for increasing a plant's ability to grow under hyperosmotic stress by overexpressing phospholipase D alpha3.

### STATEMENT OF GOVERNMENT RIGHTS

This invention was made with government support under grant no. IBN-0454866 awarded by the National Science Foundation and grant nos. 2005-35818-15253 and 2005-35318-18397 awarded by the U.S. Department of Agriculture. The government has certain rights in the invention.

### FIELD OF THE INVENTION

The present invention relates to transgenic plants and seeds with altered expression of phospholipase Dε (PLDε), and in particular to transgenic plants and seeds overexpressing PLDε. The present invention also relates to methods for producing such transgenic plants. Also included are methods for increasing a plant's ability to capture and utilize nitrogen, increasing a plant's biomass production, increasing yield of a plant, and increasing growth under hyperosmotic stress. The invention also relates to transgenic plants and seeds which overexpress phospholipase D alpha3 (PLDα3), methods for producing such transgenic plants and seeds, and methods for increasing a plant's ability to grow under hyperosmotic stress by overexpressing phospholipase D alpha3.

### BACKGROUND

Recent studies in plants indicate that phospholipase D (PLD) and its lipid product phosphatidic acid (PA) play important roles in plant response to various stresses (Wang, X. (2005) Plant Physiol. 139, 566-573; Wang, X., Devaiah, S. P., Zhang, W. & Welti, R. (2006) Prog Lipid Res. 45, 250-278). PLD is a major family of phospholipases in plants, and the Arabidopsis genome contains 12 PLDs that are classified into six types, α (3), β (2), γ(3), δ, ε, and ζ (2) (Qin, C. & Wang, X. (2002) Plant Physiol. 128, 1057-1068). PLDα1 plays a role in signaling abscisic acid regulation of stomatal movement whereas PLDδ is involved in H₂O₂ response and freezing tolerance (Zhang, W., Wang, C., Qin, C., Wood, T., Olafsdottir, G., Welti. R. & Wang, X. (2003) Plant Cell 15, 2285-2295; Zhang, W., Qin, C., Zhao, J. & Wang, X. (2004) Proc. Natl. Acad. Sci. USA. 101, 9508-9513; Mishra, G., Zhang, W., Deng, F., Zhao, J. & Wang, X. (2006) Science 312, 264-266; Li, W., Li, M., Zhang, W. & Wang, X. (2004) Nat Biotechnol. 22, 427-433). PLDζs play a role in root hair patterning, root development in response to phosphate starvation and auxin (Ohashi, Y., Oka, A., Rodrigues-Pousada, R., Possenti, M., Rubert, I., Morelli, G. & Aoyama, T. (2003) Science 300, 1427-1430; Cruz-Ramirez, A., Oropeza-Aburto, A., Razo-Hernandez, F., Ramirez-Chavez, E. & Herrera-Estrella, L. (2006) Proc. Natl. Acad. Sci. USA. 103, 6765-6770; Li, M., Qin, C., Welti, R. & Wang, X. (2006) Plant Physiol. 140, 761-770; Li, G. & Xue, H. W. (2007) Plant Cell 19, 281-295). Biochemical characterization of the plant PLD family has begun to shed light on the functional heterogeneity of these enzymes. Most plant PLDs have the Ca²⁺/phospholipids-binding C2 domain, whereas two PLDζs contain the phosphoinositide-interacting pleckstrin homology (PH) and Phox homology (PX) domains. PLDα1, β1, γ1, γ2, δ, and ζ1 display distinguishable requirements for Ca²⁺, polyphosphoinositides (PPI), or fatty acids, and substrate selectivity. These properties suggest that individual PLD is regulated differently in the cell, and the activity of PLDα1, δ, and ζ are stimulated in response to specific stimuli. Of the 12 PLDs in Arabidopsis, PLDε encodes a unique protein. It has the C2 structural fold, but contains no acidic residues in the C2 domain that are involved in Ca²⁺ binding (Qin, C. & Wang, X. (2002) Plant Physiol. 128, 1057-1068). Phylogenic analysis of the Arabidopsis and rice PLD families suggests that PLDε of all the C2-PLDs is most closely related to the PX/PH-PLDs. By comparison, mammals have no C2-PLDs, but have two PH/PX PLDs that are closely related to PLDζs.

PLDα3 is another unique member of the phospholipase D family. The PLDα group has three members, of which PLDα1 and α2 are very similar, sharing about 93% similarity in deduced amino acid sequences, whereas PLDα3 is more distantly related to them, sharing about 70% amino acid sequence similarity to each of the other two PLDαs. Furthermore, the coding region of PLDα3 contains three introns, whereas the coding regions of PLDα1 and PLDα2 are interrupted by two introns (Qin and Wang 2002).

A plant's traits, such as its biochemical, developmental, or phenotypic characteristics, may be controlled through a number of cellular processes. Strategies for manipulating traits by altering a plant cell's expression of a protein or a transcription factor can therefore result in plants and crops with new and/or improved commercially valuable properties.

### SUMMARY OF THE INVENTION

It is one embodiment of the present invention to provide a transgenic plant with altered phospholipase D epsilon (PLDε) expression relative to the corresponding wild-type plant. PLDε can be either overexpressed or underexpressed.

It is another embodiment of the present invention to provide a transgenic seed with altered phospholipase D epsilon (PLDε) expression relative to the corresponding wild-type seed. PLDε can be either overexpressed or underexpressed.

In another embodiment, the present invention relates to a method of producing a transgenic plant, which overexpresses PLDε, comprising introducing an expression construct that comprises a polynucleotide encoding a phospholipase D epsilon (PLDε) polypeptide operably linked to a promoter which is capable of overexpressing the polypeptide into a plant cell to produce a transformed plant cell; and producing a transgenic plant from the transformed plant cell.

It is still another embodiment of the present invention to provide a method for increasing a plant's ability to capture and utilize nitrogen comprising overexpressing a phospholipase D epsilon (PLDε) in the plant.

In another embodiment, the present invention relates to a method for increasing a plant's biomass production comprising overexpressing a phospholipase D epsilon (PLDε) in the plant.

Yet another embodiment of the present invention is a method for increasing yield of a plant compared to the yield of corresponding wild type plants, comprising overexpressing the phospholipase D epsilon (PLDε) in the plant.

In another embodiment, the present invention relates to a method for increasing a plant's ability to grow under hyperosmotic stress conditions compared to a corresponding wild-type plant, wherein the plant overexpresses a phospholipase D epsilon (PLDε).

It is another embodiment of the present invention to provide transgenic plants which overexpress phospholipase D alpha3 (PLDα3) relative to the corresponding wild-type plant.

It is yet another embodiment to provide transgenic seeds which overexpress phospholipase D alpha3 (PLDα3) relative to the corresponding wild-type plant.

In still another embodiment, the present invention relates to a method of producing a transgenic plant, which overexpresses phospholipase D alpha3 (PLDα3), comprising introducing an expression construct that comprises a polynucleotide encoding a phospholipase D alpha3 (PLDα3) polypeptide operably linked to a promoter which is capable of overexpressing the polypeptide into a plant cell to produce a transformed plant cell; and producing a transgenic plant from the transformed plant cell.

Yet another embodiment of the present invention is the provision of a method for increasing a plant's ability to grow under hyperosmotic stress conditions, the method comprising overexpressing a phospholipase D alpha3 (PLDα3) in the plant.

Other objects and features will be in part apparent and in part pointed out hereinafter.

### BRIEF DESCRIPTION OF THE DRAWINGS

FIG. 1 depicts alterations of PLDε expression and their effects on Arabidopsis growth. (A) T-DNA insertion site in PLDε gene. Exons are shown as white boxes. (B) PLDε transcript in PLDε-1 and WT separated on a 1% agarose gel after RT-PCR from total leaf RNA. UBQ10 was used as a control. (C) Immunoblotting of PLDε in 35S::PLDε-HA plants. Proteins (30 mg/lane) were separated by 8% SDS-PAGE and blotted with HA antibody and visualized by alkaline phosphatase conjugated with secondary anti-mouse antibody. (D) Growth phenotype. PLDε-OE, KO, and WT plants were grown in a growth chamber and fertilized with 200 ppm N once a week. (E) Growth competition of PLDε-altered and WT plants under low fertilizer and low light (20 mmol m-2 s-1) conditions. (F) Rosette dry weight as affected by N levels. High or Low N refer to plants that were watered with the fertilizer once a week or one time in the entire life cycle, respectively. Values are means±SD (n=30). * is significantly different at P<0.05, as compared to WT based on Student's t-test. (G) Leaf size of five-week-old plants grown in well fertilized soil. Values are means±SD (n=40). (H) Cell sizes of expanded leaves from 5-week-old plants. Values are means±SD (n=60) from three independent experiments. (I) Seed yield. Seeds were collected from individual plant. Values are means±SD (n=10) from one of three independent experiments.

FIG. 2 depicts the effect of PLDε alterations on root growth and nitrogen use efficiency. (A) Four-day-old seedlings were transferred to Murashige and Skoog (MS) containing 6 mM N for 12 days. (B) Four-day-old seedlings were transferred to MS with different N levels: 0.6, 6, and 60 mM N, or MS with 6 mM N containing 0.15% 1-butanol or 0.15% 2-butanol. Root length and number were measured 6 days after transfer. Values are means±SD (n=30). (C) Root hair length of seedlings germinated in MS with 0.1 mM N for 7 days. Values are means±SD (n=20). (D) Biomass accumulation of four-week-old seedlings as affected by different N levels. Values are means±SD (n=30).

FIG. 3 depicts changes in PA and biochemical properties of PLDε. (A) Leaf PA content from 4-week-old, soil-grown plants under the high N condition. Values are means±SE (n=5) and each replicate contained 6 leaves from 6 individual plants. (B) Contents of phosphatidyglycerol (PG), mongalactosyldiacylglycerol (MGDG), digalactosyldiacylglycerol (DGDG), phosphatidylcholine (PC), phosphatidylethanolamine (PE), and phosphatidic acid (PA) in roots from seedlings grown in MS containing 2 mM nitrogen. Lipids were extracted from roots 3 weeks after transfer from 60 mM N medium. Values are means±SE (n=4), and each replicate contained at least 20 seedlings. (C) Subcellular association of PLDε in comparison to PLDα2. Soluble (S) and microsomal (M) fractions were the supernatant and pellet, respectively, of the 100,000 g centrifugation of 6,000 g supernatant. The plasma membrane (PM) and intracellular membrane (IM) fractions were isolated by two-phase partitioning. The proteins were made visible by alkaline phosphatase conjugated secondary anti-mouse antibody. 30 mg/lane was loaded for soluble proteins, but 5 mg/lane with membrane fractions. (D) PLDε and α2 reaction conditions. The purified PLDε-HA and PLDα2-HA were used for PLD activity assay under PLDα1, β, δ, and ζ reaction assay conditions. Values are means±SD (n=3). (E) PLDε and α2 activity toward different phospholipids. The same amount of fluorescence-labeled lipids, including NBD-PC, -PE, -PG, or -PS, were incubated with equal amount of purified PLDε under the PLDα1 reaction conditions. Vector control was the reaction using protein from vector transformed plants by the same purification procedures.

FIG. 4 depicts changes in expression of nitrate transporters, PLDε, S6K1, and CDKA;1. (A) The expression levels of PLDε and nitrate transporters NRT1.1 and NRT2.1 under different N conditions. Seedlings grown in 60 or 0.6 mM N for 10 days, or 5-day-old seedlings on 60 mM N were transferred to 0.6 mM N for 5 days (60→0.6 mM). The transcript level was quantified by real-time PCR normalized by UBQ10. Values are means±SD (n=3). (B) Transcript levels of S6K1 and CDKA;1 under a N-rich growth condition. Values are means±SD (n=3).

FIG. 5 depicts S6K levels under different N levels. (A) and (B) S6K protein levels detected by immunoblotting with anti-p70 S6K and anti-phospho-p70 S6K (Thr389) antibodies, respectively. Four-day-old seedlings were transferred to MS plates with the indicated N levels for 10 days. The same amount of proteins (12 mg/lane) was separated by 10% SDS-PAGE and transferred to a nitrocellulose membrane. (C) S6K binding to PA immobilized on filter. Lipid (10 mg) was spotted on nitrocellulose filter, and incubated with leaf proteins (1.2 mg/ml), followed by blotting with anti-human p70 S6K antibody. (D) S6K binding to liposomes composed of PA (PA:PC=1:2), PC, or PI only (650 nmol of total lipids). (E) Total S6K, but not phosphorylated S6K binding to PA:PC liposomes. Proteins were extracted from WT and PLDε-altered seedlings. Bound proteins were separated by SDS-PAGE and detected by blotting with anti-human p70 S6K antibody or anti-human p389 p70 S6K antibody.

FIG. 6 depicts a proposed model of PLDε and its derived PA in growth signaling. Under the stimulus of nutrient, PLDε and its PA activate the potential targets, TOR, PDK1, or S6K, thus promoting cell growth. PA may directly interact with S6K and may also activate S6K via TOR and/or PDK.

FIG. 7 depicts PLDα3 expression, reaction conditions, and substrate specificity. (A) Expression of PLDα3 and α1 in Arabidopsis tissues, as quantified by real time PCR normalized to UBQ10. Values are means±SD (n=3 separate samples). (B) Production of HA-tagged PLDα3 in Arabidopsis WT plants. Leaf proteins extracted from PLDα3-HA transgenic plants were separated by 8% SDS-PAGE and transferred to PVDF membrane. PLDα3-HA was visualized by alkaline phosphatase conjugated to secondary anti-mouse antibody after blotting with HA antibody. Lanes 1 through 5 show different transgenic lines carrying the PLDα3-HA overexpression construct. (C) PLDα3 activity under PLDα1, β, δ, and ζ1 assay conditions. PLDα3-HA protein was expressed and purified from Arabidopsis leaves using HA antibody affinity immunoprecipitation and was subjected to PLDα3 activity assays under PLDα1, β, δ, and ζ1 reaction conditions using ³H-PC as a substrate. Values are means±SD (n=3) of three independent experiments. (D) Quantification of the hydrolytic activity of PLDα3 toward 12-(7-nitro-2-1,3-benzoxadiazol-4-yl)amino (NBD)-PC, PE, PG, and PS. The lipid spots on TLC plates corresponding to substrates (PC, PE, PG, and PS), and product (PA) were scraped and the lipids were extracted for fluorescence measurement (excitation at 460 nm, emission at 534 nm). Vector is a negative control that refers to reactions using HA antibody immunoprecipitates from proteins of empty vector-transformed Arabidopsis plants. Values are means±SD (n=3) of three experiments.

FIG. 8 depicts T-DNA insertion mutant of PLDα3 and effects of PLDα3 alterations on seed germination under salt stress. (A) T-DNA insertion in the second exon of PLDα3; white boxes indicate exons of PLDα3. (B) Confirmation of the T-DNA insertion in pldα3-1. PCR of genomic DNA from WT and pldα3-1 using two pairs of primers: T-DNA refers to the fragment amplified using the left border primer and a PLDα3-specific primer; PLDα3 marks the fragment amplified using two PLDα3 primers, one on either side of the T-DNA insert. The presence of the T-DNA band and the lack of the PLDα3 band in pldα3-1 indicate that it is a homozygous T-DNA insertion mutant. The experiment was repeated three times under the same conditions. (C) The loss of PLDα3 transcript in pldα3-1. Reverse-transcription PCR of RNA from WT and pldα3-1, using two pairs of primers: PLDα3-specific primers detect the expression of the PLDα3 mRNA, and UBQ10 primers were used as a control to indicate the same amount of mRNA between pldα3-1 and WT. The experiment was repeated three times under same conditions. (D) to (G) Seeds were germinated in Murashige & Skoog (MS) medium containing 0 (control), 150, or 200 mM NaCl. WT=wild type, pldα3-1=PLDα3 knockout mutant, COM=PLDα3 complementation, OE=PLDα3 overexpression. Values are means±SD (n=3) from one representative of three independent experiments with similar results. One hundred seeds per genotype were measured in each experiment. The photographs were taken 3 days after seeds were sown. Bar=3 mm.

FIG. 9 depicts the effects of altering PLDα3 expression on salt tolerance. (A) to (C) Changes in seedling growth under salt stress as affected by PLDα3-KO and OE. Four-day-old seedlings were transferred to MS agar plates with 0 (control), 50 or 100 mM NaCl. Primary root length was measured two weeks after transfer. Lateral roots were counted 6 days after transfer. Values are means±SD (n=15) from one representative of three independent experiments. The height of each square in the plate is 1.4 cm. * is significant at P<0.05, as compared to WT based on Student t-test, as in the following figures. (D) Seedling growth in 50 mM NaCl salt on agar plates for 3 weeks. (E) Changes in salt tolerance in soil-grown, PLDα3-altered plant. Three-week-old plants grown were irrigated with water only (control) or 100 mM NaCl solution. Photographs were taken three-week after the treatment. (F) Membrane ion leakage of PLDα3-altered and WT plants in response to salt stress. Relative conductivity (an indicator of ion leakage) of leaves was measured in plants grown in soil treated with water only (control) or 100 mM NaCl solution for two weeks. Values are means±SD (n=3) from one of three independent experiments. (G) Chlorophyll content of PLDα3-altered and WT plants in response to salt stress. Chlorophyll content of leaves was measured in plants as described at (E). Values are means±SD (n=3) from one of three independent experiments with similar results.

FIG. 10 depicts the growth of WT, PLDα3-KO and -OE under hyperosmotic stress. (A) and (B) Root and seedling phenotypes. (C) Seedling fresh weight. Seeds were germinated and grown in MS (control) or MS-agar plates containing 8% PEG. Fresh weights were measured 15 days after seeds were sown. Values are means±SD (n=10) from one of three independent experiments. At least 30 seedlings of each genotype were measured. (D) Primary root length. Five-day-old seedlings were transferred to 8% PEG in MS agar plates for 3 weeks and primary root length was measured. Values are means±SD (n=10) from one of three independent experiments. At least 30 seedlings of each genotype were measured. (E) Lateral root number. Root number was counted two weeks after five-day-old seedlings were transferred to 8% PEG in MS agar plates. Values are means±SD (n=10) from one of three independent experiments.

FIG. 11 depicts flowering time changes in PLDα3-KO and -OE plants under water deficits. (A) Flowering times of PLDα3-altered and WT plants grown under the same water deficient conditions. (B) Immunoblotting of PLDα3 level in two PLDα3-OE lines (upper panels) and the association of the PLDα3 protein level with flowering time (lower panels) under water deficit conditions. (C) and (D) Days to bolting and the number of rosette leaves in bolting plants under water deficits. Values are means±SD (n=15) from one representative of three independent experiments. (E) Number of siliques in two PLDα3-OE lines, WT plants, and plants transformed with the empty vector (Vector). Silique numbers were counted in 55-day-old plants grown under water deficit conditions. KO plants were not scored because they flowered later. Values are means±SD (n=20). (F) to (H) The expression of FT, BFT, and TSF in WT, PLDα3-KO and -OE plants. mRNA was extracted from leaves of 3-week-old plants (before inflorescence formation under well watered conditions, control) or from leaves of plants during inflorescence or flowering under water deficit (25-30% of soil water capacity). The expression levels were monitored by quantitative real time PCR normalized by comparison to Ubiquitin 10. Values are means±SD (n=3)

FIG. 12 depicts ABA content in and effect on PLDα3-altered and WT plants. (A) ABA content and the expression of ABA-responsive genes in PLDα3-altered and WT plants under water deficits. ABA content was measured by mass spectrometry and ABA-responsive genes were examined by real time PCR in 3-week-old plants during the transition from control water (90% of soil water capacity) to water deficient (25-30% of soil water capacity) conditions. The number of days refers to days without watering under the water deficit conditions. Values are means±SD (n=3 independent samples) from one of two independent experiments with similar results. * marks significance at P<0.05, as compared to WT based on Student t-test, and ^{a, or b} denote significance at P<0.05, as compared to day 0 within the same genotype. (B) and (C) effect of ABA on growth of PLDα3-altered seedlings. Seeds were germinated in MS containing 5 μM ABA. Fresh weights were measured 5 weeks after germination. Values are means±SD (n=20) from one of three experiments. (D) Water loss from detached leaves of PLDα3-altered plants. The leaves were detached from 5-week-old plants and exposed to cool white light (100 μmol m⁻²s⁻¹) at 23° C. Loss of fresh weight was used as a measure of water loss. pldα1=PLDα1 knockout mutant. Values are means±SD (n=5).

FIG. 13 depicts lipid changes in plants in response to drought stress. (A) Total lipid levels in PLDα3-altered, PLDα1-KO, and WT plants under water deficit and well-watered conditions. Four-week-old plants grown in growth chambers were not watered until RWC of leaves was about 40%. Well watered plants were used as control. Leaf lipids were extracted from four different samples and profiled by ESI-MS/MS. Values are means±SE (n=4). (B) Lipid species in PLDα3-altered and WT plants under water deficits. Values are means±SE (n=4) of four different samples. * is significant at P<0.05, as compared to WT based on Student t-test.

FIG. 14 depicts the levels of TOR expression, AGC2.1 expression, and phosphorylated S6K protein in PLDα3-altered and WT seedlings under hyperosmotic stress. (A) Expression level of TOR and AGC2.1 under salt and water deficit conditions. Four-day-old seedlings were transferred to MS agar plates containing 100 mM NaCl, or 8% PEG. Seedlings grown in MS without NaCl or PEG were used as the control condition (control). RNA was extracted from seedlings three weeks after transfer. Gene expression level was quantified by real time PCR normalized by Ubiquitin 10. Values are means±SD (n=3) from one of two experiments with similar results. (B) Level of phosphorylated S6K. Proteins were extracted from seedlings grown in the conditions as described in (A). The same amounts of proteins (12 μg/lane) were separated by 10% SDS-PAGE, and then were transferred to nitrocellulose membranes. Phosphorylated S6K was detected by immunoblotting with anti-phospho-p70 S7K (Thr389) antibody. Data were based on one of two experiments with similar results.

### DEFINITIONS

The term “plant” includes whole plants, plant organs (e.g., leaves, stems, roots, etc.), seeds and plant cells and progeny of same.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as osmotic stress tolerance or yield. Any technique can be used to measure the amount of, comparative level of, or difference in any selected chemical compound or macromolecule in the transgenic plants.

The terms “nucleic acid molecule” and “polynucleotide” are used interchangeably and refer to a polymeric form of nucleotides of any length, either deoxyribonucleotides or ribonucleotides, or analogs thereof. This term refers only to the primary structure of the molecule and thus includes double- and single-stranded DNA and RNA. It also includes known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide.

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The “native sequence” or “wild-type sequence” of a gene is the polynucleotide sequence that comprises the genetic locus corresponding to the gene, e.g., all regulatory and open-reading frame coding sequences required for expression of a completely functional gene product as they are present in the wild-type genome of an organism. The native sequence of a gene can include, for example, transcriptional promoter sequences, translation enhancing sequences, introns, exons, and poly-A processing signal sites. It is noted that in the general population, wild-type genes may include multiple prevalent versions that contain alterations in sequence relative to each other and yet do not cause a discernible pathological effect. These variations are designated “polymorphisms” or “allelic variations.”

As used herein, the term “expression cassette” refers to a molecule comprising at least one coding sequence operably linked to a control sequence which includes all nucleotide sequences required for the transcription of cloned copies of the coding sequence and the translation of the mRNAs in an appropriate host cell.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein. Full-length proteins, analogs, mutants and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular polypeptide may be obtained as an acidic or basic salt, or in neutral form. A polypeptide may be obtained directly from the source organism, or may be recombinantly or synthetically produced.

The phrase “open reading frame” or “coding sequence” refers to a nucleotide sequence that encodes a polypeptide or protein. The coding region is bounded in eukaryotes, on the 5′ side by the nucleotide triplet “ATG” that encodes the initiator methionine and on the 3′ side by one of the three triplets which specify stop codons (i.e., TAA, TAG, and TGA).

By “transgenic plant” is meant a plant into which one or more exogenous polynucleotides have been introduced. The transgenic plant therefore exhibits altered structure, morphology or biochemistry as compared with a progenitor plant which does not contain the transgene, when the transgenic plant and the progenitor plant are cultivated under similar or equivalent growth conditions. Such a plant containing the exogenous polynucleotide is referred to herein as an R₁ generation transgenic plant. Transgenic plants may also arise from sexual cross or by selfing of transgenic plants into which exogenous polynucleotides have been introduced. Such a plant containing the exogenous nucleic acid is also referred to herein as an R₁ generation transgenic plant. Transgenic plants which arise from a sexual cross with another parent line or by selfing are “descendants or the progeny” of an R₁ plant and are generally called Fⁿ plants or Sⁿ plants, respectively, with n meaning the number of generations.

“Transfection” is the term used to describe the introduction of foreign material such as foreign DNA into eukaryotic cells. It is used interchangeably with “transformation” and “transduction” although the latter term, in its narrower scope refers to the process of introducing DNA into cells by viruses, which act as carriers. Thus, the cells that undergo transfection are referred to as “transfected,” “transformed” or “transduced” cells.

The term “plasmid” as used herein, refers to an independently replicating piece of DNA. It is typically circular and double-stranded.

The term “vector” refers to a DNA molecule into which foreign fragments of DNA may be inserted. Generally, they contain regulatory and coding sequences of interest. The term vector includes but is not limited to plasmids, cosmids, phagemids, viral vectors and shuttle vectors.

“PLD” is an abbreviation for phospholipase D.

“KO” is an abbreviation for knock-out.

“OE” is an abbreviation for overexpressed.

“WT” is an abbreviation for wild type.

“ABA” is an abbreviation for abscisic acid.

“DAG” is an abbreviation for diacylglycerol.

“NBD” is an abbreviation for 1-oleoyl, 2-12[(7-nitro-2-1,3-benzoxadiazol-4-yl)amino]dodecanoyl.

“PA” is an abbreviation for phosphatidic acid.

“PC” is an abbreviation for phosphatidylcholine.

“PE” is an abbreviation for phosphatidylethanolamine.

“PI” is an abbreviation for phosphatidylinositol.

“PS” is an abbreviation for phosphatidylserine.

“PtdBut” is an abbreviation for phosphatidylbutanol.

### DETAILED DESCRIPTION OF THE INVENTION

Nitrogen is an important and limiting nutrient for plant growth. Increasing the amounts of nitrogen fertilizers that are being applied to crop fields can cause environmental damage and increase energy costs for crop production. Advantageously, it has been discovered that overexpressing phospholipase Dε (PLDε) in plants can increase their nitrogen utilization, and thereby increase biomass production and plant yield. Furthermore, it has been discovered that overexpressing phospholipase Dα3 (PLDα3) in plants can increase their ability to grow under hyperosmotic stress conditions, such as drought and high salinity.

Accordingly, the present invention relates to transgenic plants overexpressing phospholipase Dε relative to corresponding wild-type plants. In one embodiment, the phospholipase Dε nucleic acid sequence is derived from a monocotyledonous or dicotyledonous plant. Preferably, the phospholipase Dε is a nucleic acid sequence from Arabidopsis thaliana. More preferably, the PLDε nucleic acid sequence comprises the sequence of SEQ ID NO: 1. The present invention also relates to transgenic plants overexpressing phospholipase Dα3 relative to corresponding wild-type plants. In one embodiment, the phospholipase Dα3 nucleic acid sequence is derived from a monocotyledonous or dicotyledonous plant. Preferably, the phospholipase Dα3 is a nucleic acid sequence from Arabidopsis thaliana. More preferably, the PLDα3 nucleic acid sequence comprises the sequence of SEQ ID NO: 2. Also encompassed by the present invention are nucleotide sequences biologically and functionally equivalent to the phospholipase Dε and phospholipase Dα3 disclosed herein that encode conservative amino acid changes within the amino acid sequences thereby producing “silent” changes. Such nucleotide sequences contain corresponding base substitutions based on the genetic code compared to the nucleotide sequence of PLDε or PLDα3. Substitutes for an amino acid within the enzyme sequence disclosed herein are selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

In some embodiments, the nucleic acid biologically and functionally equivalent to PLDε nucleic acid has a nucleotide sequence with at least about 70% homology to SEQ ID NO: 1. Similarly, in some embodiments the nucleic acid biologically and functionally equivalent to PLDα3 nucleic acid has a nucleotide sequence with at least about 70% homology to SEQ ID NO: 2. As used herein, “percent homology” of two amino acid sequences or of two nucleic acids can be determined using, e.g., the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into NBLAST and XBLAST programs of Altschul et al. (J. Mol. Biol. 215:403-410, 1990). BLAST nucleotide searches are performed with the NBLAST program, score=100, wordlength=12, to obtain nucleotide sequences homologous to a nucleic acid molecule encoding PLDε. BLAST protein searches are performed with the XBLAST program, score=50, wordlength=3, to obtain amino acid sequences homologous to a reference polypeptide. To obtain gapped alignments for comparison purposes, Gapped BLAST is utilized, e.g., as described in Altschul et al. (Nucleic Acids Res. 25:3389-3402, 1997). When utilizing BLAST and Gapped BLAST programs, the default parameters of the respective programs (e.g. XBLAST and NBLAST) are used. See, for example, www.ncbi.nlm.nih.gov. Thus, in some embodiments, the polynucleotide biologically and functionally equivalent to a PLDε nucleic acid has at least about 75% homology to SEQ ID NO: 1. In a preferred embodiment, the polynucleotide biologically and functionally equivalent to a PLDε nucleic acid has at least about 80% homology to SEQ ID NO: 1, and more preferably, the polynucleotide has at least about 85%, at least about 90%, at least about 95%, at least about 97%, or at least about 99% homology to SEQ ID NO: 1. In other embodiments, the polynucleotide biologically and functionally equivalent to a PLDα3 nucleic acid has at least about 75% homology to SEQ ID NO: 2. In a preferred embodiment, the polynucleotide biologically and functionally equivalent to a PLDα3 nucleic acid has at least about 80% homology to SEQ ID NO: 2, and more preferably, the polynucleotide has at least about 85%, at least about 90%, at least about 95%, at least about 97%, or at least about 99% homology to SEQ ID NO: 2.

In other embodiments, a PLDε nucleic acid encodes a polypeptide biologically and functionally equivalent to a PLDε polypeptide, wherein the polypeptide has at least about 70% homology to a polypeptide encoded by SEQ ID NO: 1. In one particular embodiment, the polypeptide has at least about 75% homology to a polypeptide encoded by SEQ ID NO: 1, and more preferably, the polypeptide has at least about 80%, at least about 85%, at least about 90%, at least about 95%, at least about 97%, or at least about 99% homology to a polypeptide encoded by SEQ ID NO: 1. In other embodiments, a PLDα3 nucleic acid encodes a polypeptide biologically and functionally equivalent to a PLDα3 polypeptide, wherein the polypeptide has at least about 70% homology to a polypeptide encoded by SEQ ID NO: 2. In one particular embodiment, the polypeptide has at least about 75% homology to a polypeptide encoded by SEQ ID NO: 2, and more preferably, the polypeptide has at least about 80%, at least about 85%, at least about 90%, at least about 95%, at least about 97%, or at least about 99% homology to a polypeptide encoded by SEQ ID NO: 2.

Exemplary of a polynucleotide encoding a PLDε polypeptide or a PLDα3 polypeptide, or encoding a polypeptide having at least about 70%, at least about 75%, at least about 80%, at least about 85%, at least about 90%, at least about 95%, at least about 97%, or at least about 99% homology to PLDε polypeptide or a PLDα3 polypeptide is the polynucleotide sequence represented by SEQ ID NO: 1 (PLDε nucleic acid) and SEQ ID NO: 2 (PLDα3 nucleic acid), respectively. Additional exemplary polynucleotide sequences include polynucleotide sequences encoding a polypeptide having at least about 70%, at least about 75%, at least about 80%, at least about 85%, at least about 90%, at least about 95%, at least about 97%, or at least about 99% homology to a PLDε polypeptide or a PLDα3 polypeptide and hybridizing to SEQ ID NO: 1 or SEQ ID NO: 2, respectively, under stringent conditions. Generally, stringent conditions for hybridization and washing are those under which nucleotide sequences at least about 70% homologous to each other typically remain hybridized to each other. Preferably, the conditions are such that sequences at least about 75%, more preferably at least about 80%, even more preferably at least about 85%, still more preferably at least about 90%, yet even more preferably at least about 95%, still more preferably at least about 97%, and most preferably at least about 99% homologous to each other typically remain hybridized to each other. Such stringent conditions are known to those skilled in the art and can be found in Current Protocols in Molecular Biology, 6.3.1-6.3.6, John Wiley & Sons, N.Y. (1989). A preferred, non-limiting example of stringent hybridization conditions are hybridization in 6×SSC at about 45° C., followed by one or more washes in 0.2×SSC, 0.1% SDS at 50° C.-65° C. Examples of moderate to high stringency conditions include, for example, initial hybridization in 6×SSC, 5× Denhardt's solution, 100 g/ml fish sperm DNA, 0.1% SDS, at 55° C. for sufficient time to permit hybridization (e.g., several hours to overnight), followed by washing two times for 15 min each in 2×SSC, 0.1% SDS, at room temperature, and two times for 15 min each in 0.5-1×SSC, 0.1% SDS, at 55° C., followed by autoradiography. Typically, the nucleic acid molecule is capable of hybridizing when the hybridization mixture is washed at least one time in 0.1×SSC at 50° C., preferably at 55° C., more preferably at 60° C., and still more preferably at 65° C. Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

A structural gene encoding a polypeptide comprising a catalytically active, truncated or intact PLDε or PLDα3 enzyme from other organisms such as yeast can also be used in accordance with the present invention.

The progeny of the above-described plants are also considered an embodiment of the present invention, as are plant cells or transformed plant cells. Cultures of those plant cells are also contemplated. Plants produced from seeds having introduced DNA are also embodiments of the present invention.

In one embodiment, the plants that overexpress phospholipase Dε or phospholipase Dα3 are monocotyledonous plants (monocots). In another embodiment, the plants which overexpress PLDε are dicotyledonous plants (dicots). In still another embodiment, the plants which overexpress phospholipase Dα3 are dicots. In some embodiments, the monocot plant is selected from corn, rice, wheat, barley, oat, rye, buckwheat, sugar cane, onion, yam, sweet potato, banana, date, bamboo and pineapple. In preferred embodiments, the monocot plant is selected from corn, rice, wheat, sugar cane, banana, barley and oat. Even more preferably, the monocot plant is selected from corn, rice and wheat. In other embodiments, the dicot plant is selected from the group consisting of cotton, soybean, canola, bean, lentils, peanut, sunflower, broccoli, alfalfa, flax, cabbage, olive, almond, coffee, tea, clover, carrot, strawberry, raspberry, orange, apple, cherry, plum, grape, potato, tomato, parsley, coriander, dill, fennel, and Arabidopsis. Preferably, the dicot is selected from cotton, soybean, bean, lentil, peanut, alfalfa and sunflower. More preferably, the dicot plant is selected from cotton and soybean.

Arabidopsis thaliana, a dicot, is generally used as a model for genetics and metabolism in plants. Arabidopsis has a small genome, and well-documented studies are available. It is easy to grow in large numbers and mutants defining important genetically controlled mechanisms are either available, or can be readily obtained. Various methods to introduce and express isolated homologous genes are available (see Koncz et al., editors, Methods in Arabidopsis Research (1992) World Scientific, New Jersey N.J., in “Preface”). Because of its small size, short life cycle, obligate autogamy and high fertility, Arabidopsis is also a choice organism for the isolation of mutants and studies in morphogenetic and development pathways, and control of these pathways by transcription factors.

In another embodiment, the present invention relates to transgenic seeds which overexpress PLDε relative to corresponding wild-type seeds. In one embodiment, the phospholipase Dε nucleic acid sequence is derived from a monocotyledonous or dicotyledonous plant. Preferably, the phospholipase Dε is a nucleic acid sequence from Arabidopsis thaliana. More preferably, the PLDε nucleic acid sequence comprises the sequence of SEQ ID NO: 1.

In still another embodiment, the present invention relates to transgenic seeds which overexpress phospholipase Dα3 relative to corresponding wild-type seeds. In one embodiment, the phospholipase Dα3 nucleic acid sequence is derived from a monocotyledonous or dicotyledonous plant. Preferably, the phospholipase Dα3 is a nucleic acid sequence from Arabidopsis thaliana. More preferably, the PLDα3 nucleic acid sequence comprises the sequence of SEQ ID NO: 2.

The seeds can be either seeds from monocots or dicots as described above. Preferably, the seeds from monocot plant are selected from corn, rice, wheat, sugar cane, banana, barley and oat seeds. Even more preferably, the monocot plant seed is selected from corn, rice and wheat seeds. In another preferred embodiment, the dicot seeds are selected from cotton, soybean, bean, lentil, peanut, alfalfa and sunflower seeds. More preferably, the dicot plant seeds are selected from cotton and soybean seeds. Another embodiment of the present invention is a seed resulting from a cross of a plant having introduced DNA, as described above, with a nurse cultivar.

In one embodiment, the PLDε overexpression in transgenic plants and/or seeds of the present invention is at least 110% of the expression in corresponding wild-type plants or seeds. Preferably, the PLDε overexpression in transgenic plants and/or seeds is at least 150% of the expression in corresponding wild-type plants or seeds, and even more preferably, it is at least 200%.

In another embodiment, the overexpression of phospholipase Dα3 in transgenic plants and/or seeds of the present invention is at least 110% of the expression in corresponding wild-type plants or seeds. Preferably, the PLDα3 overexpression in transgenic plants and/or seeds is at least 150% of the expression in corresponding wild-type plants or seeds, and even more preferably, it is at least 200%.

In order to overexpress PLDε or PLDα3 in plants, recombinant DNA vectors suitable for transformation of plant cells are prepared. Techniques for transforming a wide variety of higher plant species are well known, and are described further below as well as in the technical and scientific literature. See, for example, Weising et al (1988) Ann. Rev. Genet. 22:421-477. A variety of methods have been developed to operatively link DNA to vectors via complementary cohesive termini or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

Alternatively, synthetic linkers containing one or more restriction endonuclease sites can be used to join the DNA segment to the plant integrating vector. The synthetic linkers are attached to blunt-ended DNA segments by incubating the blunt-ended DNA segments with a large excess of synthetic linker molecules in the presence of an enzyme that is able to catalyze the ligation of blunt-ended DNA molecules, such as bacteriophage T4 DNA ligase. Thus, the products of the reaction are DNA segments carrying synthetic linker sequences at their ends. These DNA segments are then cleaved with the appropriate restriction endonuclease and ligated into a plant integrating vector that has been cleaved with an enzyme that produces termini compatible with those of the synthetic linker. Synthetic linkers containing a variety of restriction endonuclease sites are commercially available from a number of sources including New England BioLabs, Beverly, Mass.

In one embodiment, the DNA sequence coding for phospholipase Dε or phospholipase Dα3, such as a cDNA sequence encoding the full-length PLDε or the full-length PLDα3 protein, is preferably combined with transcriptional and translational initiation regulatory sequences which will direct the transcription of the sequence from the gene in the intended tissues of the transgenic plant.

In construction of recombinant expression cassettes of the invention, a plant promoter which directs expression of the gene in all tissues of a regenerated plant is used. Such promoters are referred to as “constitutive” promoters and are active under most environmental conditions and states of development or cell differentiation. Examples of constitutive plant promoters which can be used include: the cauliflower mosaic virus (CaMV) 35S promoter, which confers constitutive, high-level expression in most plant tissues (see, for example, Odell et al. (1985) Nature 313: 810-812); the nopaline synthase promoter (An et al. (1988) Plant Physiol. 88: 547-552); the T-DNA mannopine synthetase promoter (e.g., the 1′- or 2′-promoter derived from T-DNA of Agrobacterium tumafaciens), and the octopine synthase promoter (Fromm et al. (1989) Plant Cell 1: 977-984). Preferably, the constitutive promoter is the cauliflower mosaic virus (CaMV) 35S promoter.

In another embodiment, a promoter that causes the transcription factor to be expressed in response to environmental, tissue-specific or temporal signals can be used. A variety of plant gene promoters are known to regulate gene expression in response to environmental, hormonal, chemical, developmental signals, and in a tissue-active manner; many of these may be used for expression of a TF sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can be employed favorably to promote expression of a polynucleotide of the invention in a transgenic plant or cell of interest. For example, tissue specific promoters include: the E4 promoter (Cordes et al. (1989) Plant Cell 1:1025), the E8 promoter (Deikman et al. (1988) EMBO J. 7: 3315), the kiwifruit actinidin promoter (Lin et al. (1993) PNAS 90: 5939), the 2A11 promoter (Houck et al., U.S. Pat. No. 4,943,674), and the tomato pZ130 promoter (U.S. Pat. Nos. 5,175,095 and 5,530,185); seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11: 651-662), root-specific promoters, such as ARSK1, and those disclosed in U.S. Pat. Nos. 5,618,988, 5,837,848 and 5,905,186, epidermis-specific promoters, including CUT1 (Kunst et al. (1999) Biochem. Soc. Trans. 28: 651-654), pollen-active promoters such as PTA29, PTA26 and PTA13 (U.S. Pat. No. 5,792,929), promoters active in vascular tissue (Ringli and Keller (1998) Plant Mol. Biol. 37: 977-988), flower-specific (Kaiser et al. (1995) Plant Mol. Biol. 28: 231-243), pollen (Baerson et al. (1994) Plant Mol. Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al. (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) Plant Mol. Biol. 38: 817-825) and the like. Additional promoters are those that elicit expression in response to nitrate (Back et al. (1991) Plant Mol. Biol. 17: 9), hormones (Yamaguchi-Shinozaki et al. (1990) Plant Mol. Biol. 15: 905; Kares et al. (1990) Plant Mol. Biol. 15: 905), heat (Ainley et al. (1993) Plant Mol. Biol. 22: 13-23), light (e.g., the pea rbcS-3A promoter, Kuhlemeier et al. (1989) Plant Cell 1: 471-478, and the maize rbcS promoter, Schaffner and Sheen (1991) Plant Cell 3: 997-1012); wounding (e.g., wunl, Siebertz et al. (1989) Plant Cell 1: 961-968); pathogens (such as the PR-1 promoter described in Buchel et al. (1999) Plant Mol. Biol. 40: 387-396, and the PDF1.2 promoter described in Manners et al. (1998) Plant Mol. Biol. 38: 1071-1080), and chemicals such as methyl jasmonate or salicylic acid (Gatz (1997) Annu. Rev. Plant Physiol. Plant Mol. Biol. 48: 89-108). In addition, the timing of the expression can be controlled by using promoters such as those acting at senescence (Gan and Amasino (1995) Science 270: 1986-1988); or late seed development (Odell et al. (1994) Plant Physiol. 106: 447-458). Plant promoters are well known in the art, and a skilled artisan can readily determine the most suitable promoters for use in particular plants.

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions, and/or a polyadenylation site.

The vector comprising the PLDε coding sequence or the PLDα3 coding sequence, and a promoter can also typically include a marker gene which confers a selectable phenotype on plant cells. For example, the marker can encode biocide resistance, particularly antibiotic resistance, such as resistance to kanamycin, G418, bleomycin, hygromycin, or herbicide tolerance, such as resistance to chlorosulfuron, glyphosate or glufosinate.

In one embodiment, DNA constructs can be introduced into the genome of a desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly into plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example, Horsch et al. (1984) Science 233:496-498, and Fraley et al. (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al. (1985) Science 227:1229-1231). Generally, the Agrobacterium transformation system is used to engineer dicotyledonous plants (Bevan et al. (1982) Ann. Rev. Genet 16:357-384; Rogers et al. (1986) Methods Enzymol. 118:627-641). The Agrobacterium transformation system can also be used to transform, as well as transfer, DNA to monocotyledonous plants and plant cells. (See Hernalsteen et al. (1984) EMBO J 3:3039-3041; Hooykass-Van Slogteren et al. (1984) Nature 311:763-764; Grimsley et al. (1987) Nature 325:1677-179; Boulton et al. (1989) Plant Mol. Biol. 12:31-40; and Gould et al. (1991) Plant Physiol. 95:426-434). Additional gene transfer and transformation methods include, but are not limited to, protoplast transformation through calcium-, polyethylene glycol (PEG)-mediated uptake of naked DNA (see Paszkowski et al. (1984) EMBO J 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276), and silicon carbide mediated DNA uptake (Kaeppler et al. (1990) Plant Cell Reporter 9:415-418).

A transformed plant cell, callus, tissue or plant may be identified and isolated by selecting or screening the transgenic plant for traits encoded by the marker genes present on the transforming DNA. For instance, selection may be performed by growing the transgenic plant in media containing an inhibitory amount of the antibiotic or herbicide to which the transforming gene construct confers resistance. Furthermore, transformed plants and plant cells may also be identified by screening for the activities of any visible marker genes (e.g., the β-glucuronidase, luciferase, B or Cl genes) that may be present on the recombinant nucleic acid constructs of the present invention. Such selection and screening methodologies are well known to those skilled in the art.

Physical and biochemical methods also may be used to identify plant or plant cell transformants containing the gene constructs of the present invention. These methods include but are not limited to: (1) Southern analysis or PCR amplification for detecting and determining the structure of the recombinant DNA insert; (2) Northern blot, S1 RNase protection, primer-extension or reverse transcriptase-PCR amplification for detecting and examining RNA transcripts of the gene constructs; (3) enzymatic assays for detecting enzyme activity; and (4) protein gel electrophoresis, Western blot techniques, immunoprecipitation, or enzyme-linked immunoassays. Additional techniques, such as in situ hybridization, enzyme staining, and immunostaining, also may be used to detect the presence or expression of the recombinant construct in specific plant organs and tissues. The methods for performing all these assays are well known to those skilled in the art.

Effects of PLDε or PLDα3 gene manipulation can be observed by, for example, Northern blots of the RNA (e.g., mRNA) isolated from the tissues of interest. Typically, if the amount of mRNA has increased, it can be assumed that the PLDε gene or the PLDα3 gene is being expressed at a greater rate than before. In addition, the levels of PLDε protein expressed can be measured immunochemically, i.e., ELISA, RIA, EIA and other antibody based assays well known to those of skill in the art, or by electrophoretic detection assays (either with staining or western blotting).

Transformed plant cells which are produced by any of the above transformation techniques can be cultured to regenerate a whole plant which possesses the transformed genotype and thus the desired phenotype. Such regeneration typically relies on a biocide and/or herbicide marker which has been introduced together with the PLDε coding sequence. More specifically, only the plants that carry the DNA construct containing PLDε or PLDα3 will exhibit resistance to a marker (e.g., biocide or herbicide) also contained in the DNA construct. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al., (1987) Ann. Rev. of Plant Phys. 38:467-486.

One of skill in the art will recognize that after the expression cassette is stably incorporated in transgenic plants and confirmed to be operable, it can be introduced into other plants by sexual crossing. Any of a number of standard breeding techniques can be used, depending on the species to be crossed.

In another embodiment, the present invention relates to a method for increasing a plant's ability to capture and utilize nitrogen by overexpressing phospholipase Dε in the plant. The overexpression of PLDε can be achieved by any of the transformation methods discussed above. As noted previously, it has been discovered that PLDε and its derived product phosphatidic acid (PA) play a role in regulation nitrogen acquisition. Specifically, overexpression of PLDε leads to improved nitrogen capture and utilization efficiency in plants. Accordingly, it is contemplated that overexpression of PLDε in plants would lead to better growth in soil poor in nitrogen, or can alternatively allow for a reduced use of nitrogen fertilizers. Better nitrogen utilization in plants is needed due to the use of nitrogen-based fertilizers and their ability to negatively affect the environment.

It is another embodiment of the present invention to provide a method for increasing a plant's biomass production by overexpressing PLDε in the plant. While not being bound to a theory, it is believed that the increased nitrogen capture and utilization achieved by PLDε overexpression leads to an increase in biomass production. As shown in the examples, plants overexpressing PLDε exhibit increases in seed number, seed size, leaf size, and leaf cell number. Thus, in another embodiment, the increase in biomass size includes, but is not limited to, increases in seed number, seed size, leaf size, leaf cell number, root size and a combination of two or more of these characteristics. Preferably, the seed number increase in PLDε-overexpressed plants is at least about 10% higher than the seed yield of corresponding wild-type plants, and more preferably it is at least about 25% higher. In another preferred embodiment, the leaf size increase in PLDε-overexpressed plants is at least about 50% higher than the leaf size of corresponding wild-type plants, and more preferably it is at least about 85% higher.

The ability to increase the biomass or size of a plant has several important commercial applications. Crop species can be generated that produce higher yields on larger cultivars, particularly those in which the vegetative portion of the plant is edible. For example, increasing plant leaf biomass may increase the yield of leafy vegetables for human or animal consumption. Additionally, increasing leaf biomass can be used to increase production of plant-derived pharmaceutical or industrial products. By increasing plant biomass, increased production levels of the products can be obtained from the plants. Furthermore, it may be desirable to increase crop yields of plants by increasing total plant photosynthesis. An increase in total plant photosynthesis is typically achieved by increasing leaf area of the plant. Additional photosynthetic capacity can be used to increase the yield derived from particular plant tissue, including the leaves, roots, fruits or seed. In addition, the ability to modify the biomass of the leaves may be useful for permitting the growth of a plant under decreased light intensity or under high light intensity. Modification of the biomass of another tissue, such as roots, can be useful to improve a plant's ability to grow under harsh environmental conditions, including drought or nutrient deprivation, because the roots may grow deeper into the ground. Increased biomass can also be a consequence of some strategies for increased tolerance to stresses, such as drought stress. Early in a stress response plant growth (e.g., expansion of lateral organs, increase in stem girth, etc.) can be slowed to enable the plant to activate adaptive responses. Growth rate that is less sensitive to stress-induced control can result in enhanced plant size, particularly later in development.

For some ornamental plants, the ability to provide larger varieties is highly desirable. For many plants, including fruit-bearing trees, trees that are used for lumber production, or trees and shrubs that serve as view or wind screens, increased stature provides improved benefits in the forms of greater yield or improved screening.

By increasing a plant's biomass production, the plant yield is also increased. Accordingly, the present invention provides a method for increasing a yield of a plant by overexpressing PLDε in the plant. By way of example and not of limitation, increased leaf size is relevant for cultivation of leafy plants, such as tea; increased root size is relevant to cultivation of plants, such as potato, and the like. In one embodiment, the increased plant yield comprises increased seed yield. Preferably, the increased seed yield is selected from the increased number of filled seeds, increased total seed weight and a combination thereof.

In one embodiment, the PLDε sequence overexpressed in plants for purposes of increasing nitrogen utilization, increasing biomass and increasing yield is the PLDε from Arabidopsis. In another embodiment, the PLDε has a nucleic acid sequence of SEQ ID NO: 1. In still another embodiment, the PLDε overexpression is at least 150% of the expression in corresponding wild-type plants or seeds. Preferably, the PLDε overexpression is at least 200% of the expression in corresponding wild-type plants or seeds, and even more preferably, it is more than 200%.

In another embodiment, the present invention relates to a method for increasing a plant's ability to grow under hyperosmotic stress conditions compared to a corresponding wild-type plant, wherein the plant overexpresses a phospholipase D epsilon (PLDε). Preferably, the PLD epsilon nucleic acid sequence comprises the sequence of SEQ ID NO: 1. As shown in the examples, ablation of PLDε made Arabidopsis more sensitive to hyperosmotic stress, such as salt, sorbitol, and PEG treatments, whereas overexpression of PLDε allowed plants to grow faster and accumulate more biomass.

In still another embodiment, the present invention relates to a method for increasing a plant's ability to grow under hyperosmotic stress conditions compared to a corresponding wild-type plant, wherein the plant overexpresses a phospholipase Dα3 (PLDα3). In this embodiment, the method generally comprises overexpressing a phospholipase D epsilon (PLDε) nucleic acid sequence in the plant. The PLDε may be overexpressed in the plant according to any of the methods disclosed herein and well known to one skilled in the art. In one embodiment, the phospholipase Dα3 nucleic acid sequence is derived from a monocotyledonous or dicotyledonous plant. Preferably, the phospholipase Dα3 is a nucleic acid sequence from Arabidopsis thaliana. More preferably, the PLDα3 nucleic acid sequence comprises the sequence of SEQ ID NO: 2.

Hyperosmotic stress is characterized by decreased turgor pressure and water availability, and is one of the more important environmental stresses that limit plant growth and productivity. Plants experience hyperosmotic stress under adverse growth conditions, such as high salinity, drought, or low temperature, and respond and acclimate to hyperosmotic stress by changing gene expression, cellular metabolism, and growth patterns. Various transcription factors and different protein kinases have been implicated in mediating plant response to hyperosmotic stress (Zhu, 2002; Jonak et al., 2002). However, the biochemical and molecular mechanism by which hyperosmotic stress is perceived and transduced into a plant response is still poorly understood. Thus, in one embodiment, the hyperosmotic stress is caused by any condition that results in decreased turgor pressure in the plant and/or decreased water availability to the plant, including but not limited to, conditions such as high salinity, drought, low temperature or any combination thereof. In one preferred embodiment, the hyperosmotic stress is caused by drought. In another preferred embodiment, the hyperosmotic stress is caused by high salinity.

In another embodiment, overexpressing the phospholipase Dα3 in the plant comprises introducing into the plant an expression construct that comprises a polynucleotide encoding a phospholipase Dα3 polypeptide operably linked to a promoter which is capable of overexpressing the polypeptide.

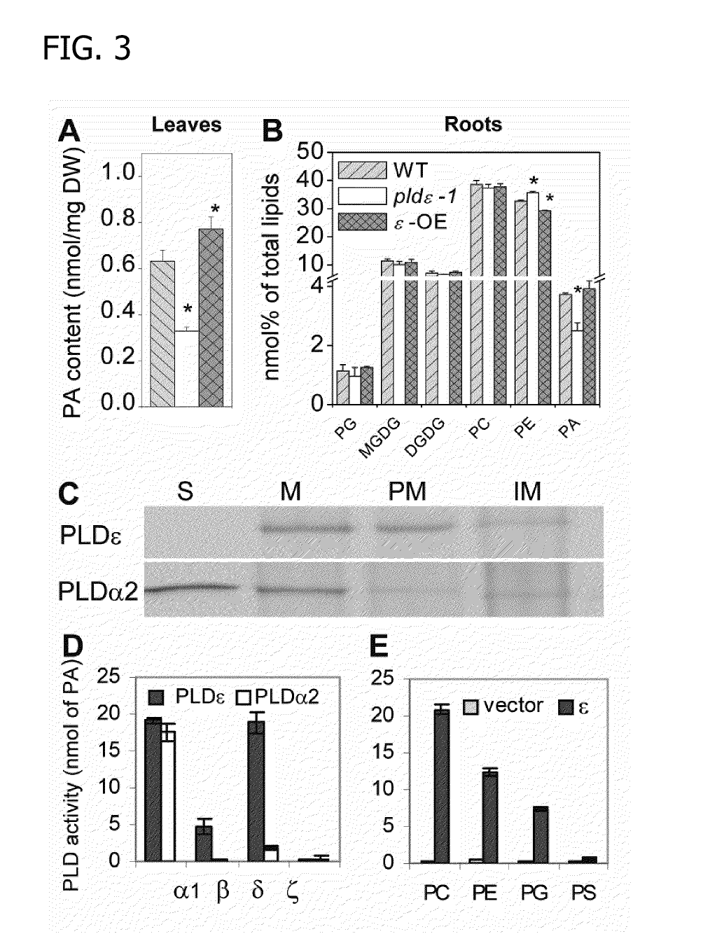

In one embodiment, increasing the plant's ability to grow under hyperosmotic stress includes but is not limited to faster growth, early flowering and/or increased biomass compared to the growth, flowering or biomass of a corresponding wild-type plant grown under the same hyperosmotic stress conditions. In one embodiment, transgenic plants overexpressing phospholipase Dα3 exhibit increased root size and/or improved root growth compared to corresponding wild-type plants grown under the same hyperosmotic stress conditions. In another embodiment, transgenic plants overexpressing phospholipase Dα3 undergo earlier flowering compared to corresponding wild-type plants grown under the same hyperosmotic stress conditions. As is known, early flowering allows plants to accelerate their life cycle, an important mechanism by which plants escape stress. As shown in the Examples and FIGS. 3-5, plants overexpressing phospholipase Dα3 grew better than wild-type plants when exposed to hyperosmotic conditions such as high salinity, presence of PEG, or limited water supply.

In other embodiments, it may be useful to underexpress PLDε. Thus, the present invention also contemplates transgenic plants and seeds which underexpress PLDε. A number of methods can be used to inhibit gene expression in plants. For instance, antisense technology can be conveniently used. To accomplish this, a nucleic acid segment from the desired gene, i.e., PLDε, is cloned and operably linked to a promoter such that the antisense strand of RNA will be transcribed. The expression cassette is then transformed into plants and the antisense strand of RNA is produced. In plant cells, antisense RNA may inhibit gene expression by preventing the accumulation of mRNA which encodes the enzyme of interest. See, e.g., Sheehy et al. (1988) Proc. Nat. Acad. Sci. USA 85:8805-8809, and Hiatt et al., U.S. Pat. No. 4,801,340.

The nucleic acid segment to be introduced generally will be substantially identical to at least a portion of the endogenous gene or genes to be repressed. The sequence, however, need not be perfectly identical to inhibit expression.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about the full length of sequence should be used, although a sequence of at least about 100 nucleotides is preferred, a sequence of at least about 200 nucleotides is more preferred, and a sequence of at least about 500 nucleotides is especially preferred. It is to be understood that any integer between the above-recited ranges is also included herein.

Catalytic RNA molecules or ribozymes can also be used to inhibit expression of PLDε gene. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloffet al. (1988) Nature 334:585-591.

Another method of suppression is sense suppression. Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al (1990) The Plant Cell 2:279-289 and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 50%-65%, but a higher identity might exert a more effective repression of expression of the endogenous sequences. Substantially greater identity of more than about 80% is preferred, though about 95% to absolute identity would be most preferred.

For sense suppression, the introduced sequence in the expression cassette, needing less than absolute identity, also need not be full length, relative to either the primary transcription product or fully processed mRNA. This may be preferred to avoid concurrent production of some plants which are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

In another embodiment, the present invention relates to transgenic plants underexpressing phospholipase Dε relative to corresponding wild-type plants or being knock-outs for phospholipase Dε. In one embodiment, the phospholipase Dε nucleic acid sequence is derived from a monocotyledonous or dicotyledonous plant. Preferably, the phospholipase Dε is a nucleic acid sequence from Arabidopsis thaliana. Such transgenic plants can be useful if it is desirable to obtain plants which are smaller than the wild-type plants.

General Methods

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-interscience; Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirety) of each of these publications are hereby incorporated herein by reference.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] Experimental Method for Gene introduction & Expression Analysis”, Yodo-sha, 1997; and the like. Gene introduction can be confirmed by methods as described herein, such as Northern blotting analysis and Western blotting analysis, or other well-known, common techniques.

Amino acid deletion, substitution or addition of the polypeptides can be carried out by a site-specific mutagenesis method which is a well known technique. One or several amino acid deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci. USA, 82, 488 (1985); Proc. Natl. Acad. Sci., USA, 81, 5662 (1984); Science, 224, 1431 (1984); PCT WO85/00817(1985); Nature, 316, 601 (1985); and the like.

Having described the invention in detail, it will be apparent that modifications and variations are possible without departing from the scope of the invention defined in the appended claims. Furthermore, it should be appreciated that all examples in the present disclosure, while illustrating the invention, are provided as non-limiting examples and are, therefore, not to be taken as limiting the various aspects of the invention so illustrated. Publications cited throughout this document are hereby incorporated herein by reference in their entirety.

### Examples

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Materials and Methods

Isolation of PLDε Knockout and Genetic Complementation. A PLDε T-DNA insert mutant was identified from SALK_{—}023603 of the Salk Arabidopsis T-DNA knockout collection (38). Seeds were obtained from the Ohio State University Arabidopsis Biological Resource Center (ABRC). The homozygous T-DNA insert mutant PLDε-1 was isolated by PCR-based screening using PLDε-specific primers and a T-DNA left border primer (SEQ ID NO: 5). The loss transcription of PLDε was confirmed by reverse transcription PCR using PLDε specific primers. To complement PLDε-1, the native PLDε gene including its own promoter region was amplified 1.5 kb upstream of the start codon and 600 bp after the stop codon and then cloned into pEC291 vector. The plasmid was transformed into PLDε-1 plants by flower dipping. Transformants were selected from hygromycin plates and confirmed by PCR.

PLDε: Plant Growth and Treatments. Plants were grown in soil under two different conditions. One set of plants was grown in growth chambers with 12-h light/12-h dark, 23/21° C., 50% humidity, 200 mmol m⁻²s⁻¹ of light intensity and watered with fertilizer (Scotts 15-5-15 Cal-Mag, 200 ppm nitrogen) once a week. The fertilizer contains 15% total nitrogen (1.2% ammonia, 11.75% nitrate, 2.05% urea), 5% available phosphate, and 15% soluble potassium.

Another set of plants was grown in the similar condition except fertilizer was applied only once to 3-week-old plants in the entire life cycle. Rosettes were dried at 80° C. for 48 hours to measure dry weight. To test N response on plates, surface-sterilized seeds were germinated in MS (1×) agar plates supplement with 3% sucrose for 4 days and then were transferred to modified MS agar plates containing 0.1, 0.6, 2, 6, 60 mM N, NO₃⁻:NH₄⁻=2:1). The lower than normal potassium ion concentration in reduced potassium nitrate media was compensated by addition of potassium chloride (Martin, T., Oswald, O. & Graham, I. A. (2002) Plant Physiol. 128, 472-481). Seedlings were grown on plates in a vertical orientation in a growth chamber under the conditions of 16-h light/8-h dark, 23/21° C., cool fluorescent white light (200 mmol m⁻² s⁻¹). Alternatively, seeds were also directly germinated in agar plates with the same conditions as described above.

RNA Extraction and Real Time PCR. Total RNA was extracted from leaves or seedlings using a CTAB method. DNA was removed from RNA by digestion with RNase-free DNasel. RNA without DNA contamination was used as a template to run reverse transcription PCR for the synthesis of cDNA using iScript kit (Bio-Rad). Quantitative real time PCR was performed with a MyiQ sequence detection system (Bio-Rad) by monitoring fluorescent labeling of double stranded DNA synthesis as described previously (Li, M., Qin, C., Welti, R. & Wang, X. (2006) Plant Physiol. 140, 761-770). The expression levels of genes were normalized by comparison to UBQ10 gene.

PLDε: Subcellular fractionation and PLD Activity Assays. Proteins were extracted from leaves of four-week old plants using chilled buffer A as described previously ((Fan, L., Zheng, S., Cui, D. & Wang, X. (1999) Plant Physiol. 119, 1371-1378), followed by centrifugation at 6,000 g for 10 min.

The supernatant was centrifuged at 100,000 g for 60 min, and the resultant supernatant and pellet were referred to as soluble and microsomal fractions, respectively. The microsomal fraction was separated into the plasma membrane and intracellular membrane fractions by two-phase partitioning as described previously (Fan, L., Zheng, S., Cui, D. & Wang, X. (1999) Plant Physiol. 119, 1371-1378). PLDs were purified from OE Arabidopsis leaves using HA-antibody affinity chromatography. The purified PLDs were assayed for activity conditions previously defined for PLDα1, β, δ, and ζ1. To assay PLD activities toward PC, PE, PG, and PS, fluorescent NBD-labeled lipids were used as substrates, and purified PLDε-HA (20 ml) was added to the reaction mixture to initiate reaction under the PLDα1 reaction conditions (25 mM Ca²⁺, 0.5 mM SDS, and 2 mM lipids). The resultant lipids were separated on TLC plates and quantified by fluorescence spectrophotometer (excitation at 460 nm, emission at 534 nm).

PLDε: Immunoblotting of PLD and S6K. Proteins were extracted from leaves or seedlings as described previously (Wang, C. & Wang, X. (2001) Plant Physiol. 127, 1102-1112). Homogenates were centrifuged at 6000 g for 10 min. For PLD-HA detection, supernatant proteins (30 mg/lane) were separated by 8% (w/v) SDS-PAGE gel, followed by transferring onto a polyvinylidene fluoride (PVDF) membrane. Membranes were blotted with anti-HA antibodies overnight and then incubated with secondary antibodies as described (6). For S6K assays, proteins (12 mg/lane) were separated by 10% SDS-PAGE and transferred to nitrocellulose membranes, followed by blotting with anti-phospho-p70 S6K (Thr389) antibody or p70 S6K antibody (Cell Signaling, Beverly, Mass.). A secondary antibody conjugated with horse-radish peroxidase was used, and after incubation with LumiGLO substrate for one min, membranes were exposed to x-ray film.

PLDε: PA-Protein Binding by Blotting and Liposomal Assays. S6K binding to lipids immobilized on a nitrocellulose filter was performed as described (Zhang, W., Qin, C., Zhao, J. & Wang, X. (2004) Proc. Natl. Acad. Sci. USA. 101, 9508-9513; Stevenson, J. M., Perera, I. Y. & Boss, W. F. (1998) J Biol Chem. 273, 22761-22767) with minor modifications. Briefly, phospholipids were immobilized on a nitrocellulose membrane. After preblotting with 3% (w/v) fatty acid-free BSA in TBST for 1 hr, the membrane was incubated with Arabidopsis proteins extracted from seedlings or leaves in a binding buffer (125 mM KCl, 25 mM Tris, pH 7.2, 1 mM DTT, 0.5 mM EDTA) at 4° C. overnight with agitation. The membrane was washed three times with TBST, and then blotted with anti-human p70 S6K antibody (1:1000). The proteins were visualized by alkaline phosphatase conjugated with secondary anti-rabbit antibody. Liposomal binding assay was based on a described method (Levine, T. P. & Munro, S. (2002) Curr Biol. 12, 695-704) with some modifications. Single class lipids or PC:PA mixture (PC:PA=2:1, molar ratio) were dried under a stream of nitrogen, and then rehydrated in a buffer with 250 mM Raffinose, 25 mM Tris (pH 7.2), and 1 mM DTT for 1 hr at room temperature. Liposomes were prepared by extrusion according to manufacturer's instruction (Avanti Polar Lipids). Liposomes were diluted three volumes in the binding buffer as described above, and centrifuged at 20,000 g for 40 min. The liposome pellet was resuspended in the binding buffer and mixed with Arabidopsis proteins (supernatant of 18,000 g, 30 min) at a final concentration of 1.2 μg/μl. After incubation at room temperature for 1 hour, liposomes were pelleted at 16,000 g for 30 min. Liposome was washed two times with the binding buffer and centrifuged again. Protein binding to liposome was detected by immunoblotting as described above.

PLDε: Leaf Area, Cell Size, and Lipid Analysis. Leaf area was measured using the equation Y=0.7888X−0.1408 (R²=0.9792), where Y is leaf area, and X is leaf width x length, generated using 40 leaves grown under the same conditions. To analyze cell size, leaf discs (0.5 cm diameter) were taken from the middle of fully expanded leaves of 5-week-old plants, and fixed in ethanol:glacial acid (3:1, v/v) for 30 min. Leaf discs were transferred sequentially to 75%, 50%, 25%, and 0% ethanol (v/v) for 15 min each. Cell sizes were measured under a microscope using the IMAGEPRO software (Media Cybernetics, Silver Spring, Md.). Lipid extraction and mass spectrometry analysis of lipids were performed as described previously (43).

Alterations of PLDε Change Cell Growth and Biomass Production and Isolation of PLDε Knockout and Genetic Complementation. A PLDε T-DNA insert mutant was identified from SALK_{—}023603 of the Salk Arabidopsis T-DNA knockout collection (Alonso et al., 2003). Seeds were obtained from the Ohio State University Arabidopsis Biological Resource Center (ABRC). PLDε homozygous T-DNA insert mutant was isolated by PCR-based screening using PLDε-specific primers: PLDε45: 5′-AGA GGG ATC CAT GGA GCT TGA AGA ACA GAA GAA G-3′ (forward) (SEQ ID NO: 3) and PLDε43: 5′-GTT AGG CCT GGT GGT TAG AAC AGG AGG AAA CA-3′ (reverse) (SEQ ID NO: 4), and a T-DNA left border primer: 5′-GCG TGG ACC GCT TGC TGC AAC T-3′ (SEQ ID NO: 5).

The loss transcription of PLDε was confirmed by reverse transcription PCR using PLDε specific primers: 5′-TAT CTT GAA CCG GGA TGG TGC AGA-3′ (forward) (SEQ ID NO: 6) and 5′-TAG GGT TTA GTG CCC ATC CTG CAA-3′ (reverse) (SEQ ID NO: 7).

To complement the PLDε knockout mutant, the native PLDε gene including its own promoter region was amplified from the 1.5 kb upstream of the start codon and 600 bp after the stop codon and then was cloned into pEC291 vector. The primers for complementation were: 5′-GAA TTC GCG TCC TCG CAT GTC TCA GGT AAA-3′ (forward) (SEQ ID NO: 8), 5′-GGA TCC TGC CCT CAT GTG TTC TTA TCA AGG ACA-3′ (reverse) (SEQ ID NO: 9). The plasmid was transformed into PLDε knockout mutant plants with the flower dipping method. The transformants were selected from hygromycin plates and were confirmed by PCR.

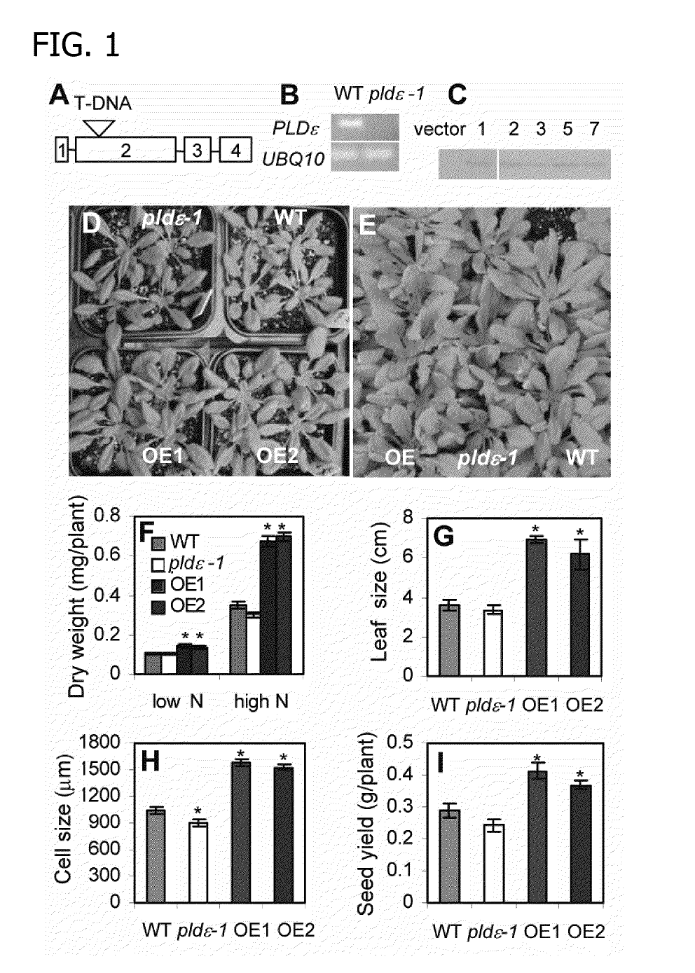

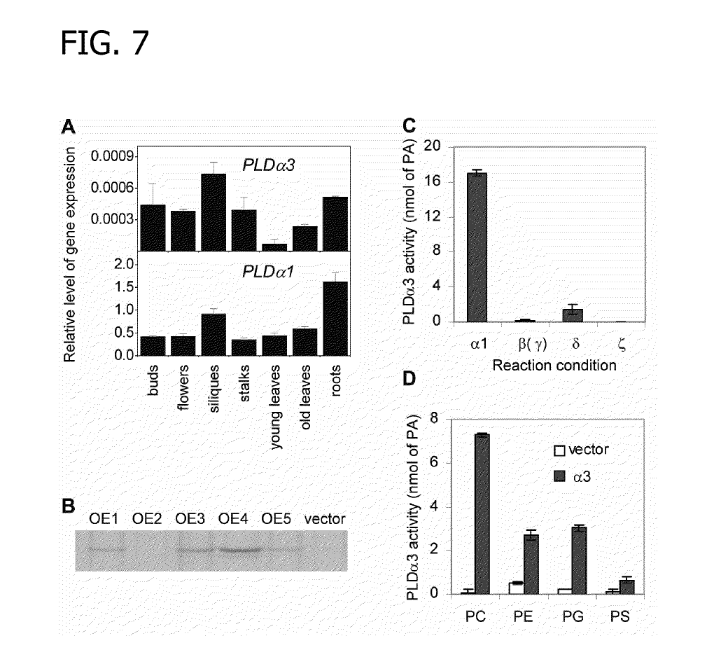

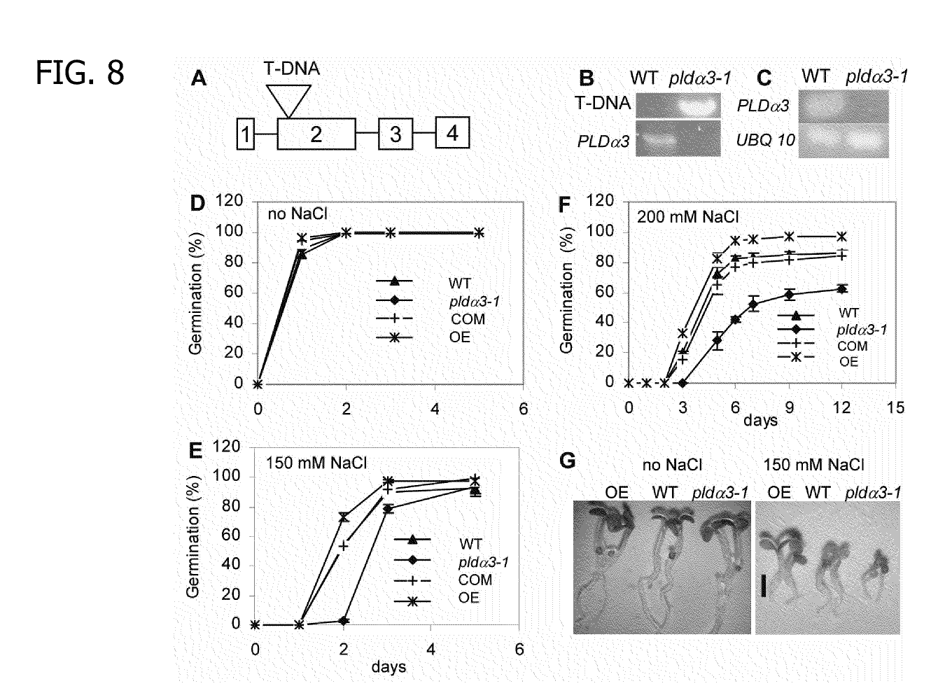

The mutant pldε-1 contained a T-DNA insertion in the second exon, 542 bp from the start codon (FIG. 1A). The mutation resulted in loss of the expression of PLDε as indicated by the absence of the detectable transcript by RT-PCR (FIG. 1B). The mutant allele co-segregated with kanamycin resistance and susceptibility in a 3:1 ratio, suggesting a single T-DNA insertion in the genome. The mutant was complemented by introducing the genomic DNA of PLDε with its own promoter. The biomass of pldε-1 was about 10% less than that of WT. The difference in biomass accumulation became larger when plants were kept under hyperosmotic stressed conditions. The knockout mutant pldε-1 accumulated only 50% or 65% of dry matter of WT in the presence of 50 mM NaCl or 100 mM sorbitol, respectively (FIG. 7A). The increased stress sensitivity was also apparent in seedlings that directly germinated in MS containing NaCl, sorbitol, or 5-8% PEG. Furthermore, pldε-1 seedlings grew slower and had shorter roots whereas OE seedlings grew more and had longer roots than WT seedlings under hyperosmotic conditions (FIG. 7B and FIG. 8).

Furthermore, PLDε was ectopically expressed in Arabidopsis under the control of the cauliflower mosaic virus 35S promoter. The coding region of PLDε was fused with a haemagglutin (HA)-tag at its C-terminus to facilitate the isolation and characterization of PLDε.

More than 20 independent PLDε-overexpressed (OE) lines were obtained, and the expression of PLDε-HA was confirmed by immunoblotting using HA-antibody (FIG. 1C).

The OE plants were tested for their growth alterations, and all lines grew larger and faster than WT and KO plants (FIG. 1D). Two OE transgenic lines were used for detailed characterization. The fresh and dry weights of rosettes of PLDε-OE were 192% and 212% of WT, respectively, after 5 weeks of growth under a well-fertilized condition (FIG. 1F).

The increase in biomass resulted from increases in leaf size (FIG. 1G) and cell size (FIG. 1H). The magnitude of increase in cell size was smaller than that of leaf area, implying that cell number also increased in PLDε-OE plants. PLDε-OE also had approximately 25% higher seed yield than WT (FIG. 1I). The enhanced growth in PLDε-OE plants was also observed under a lower fertilized condition (FIG. 1F). When OE, KO, and WT plants were planted in the same tray under a low fertilized condition, KO plants were much smaller in size and out-competed by WT and OE (FIG. 1E).

PLDα3 Knockout Mutant Isolation and Complementation. A T-DNA insert mutant in PLDα3, designated as pldα3-1, was identified from the Salk Arabidopsis T-DNA knockout collection (SALK_{—}130690) and seeds were obtained from the Ohio State University Arabidopsis Biological Resource Center (ABRC). A PLDα3 homozygous T-DNA insert mutant was isolated by PCR screening using PLDα3-specific primers: 5′-CTC GAG ATG ACG GAC CAA TTG CTG CTT CAT CG-3′ (forward primer) (SEQ ID NO: 10), 5′-ACG CCT AGA AGT AAG GAT GAT TGG AGG AAG A-3′ (reverse primer) (SEQ ID NO: 11), and a left border primer: 5′-GCG TGG ACC GCT TGC TGC AAC T-3′ (SEQ ID NO: 12). A pair of PLDα3-specific primers were used in reverse transcription PCR to confirm the PLDα3 null mutant: 5′-ATG GTT AAT GCA ACG GCA GAC GAG-3′ (forward) (SEQ ID NO: 13) and 5′-CCC GGT AAA TCG TCA TTT CGA GGA-3′ (reverse) (SEQ ID NO: 14). The PCR conditions were 95° C. for 1 min for DNA melting, 40 cycles of 95° C. for 30 s, annealing for 30 s (annealing temperature was based on the melting points of the specific primers), and 72° C. for 30 s for DNA extension. Finally, the reaction was set at 72° C. for 10 min. For complementation of the PLDα3 knockout mutant, the native PLDα3 gene including its own promoter region was amplified from 1.5 kb upstream of the start codon and 600 bp after the stop codon and then was cloned into the pEC291 vector. The primers for PLDα3 complementation were: 5′-CTG CAG GTA AGA TTC ACA AAA TTG GTG TAA TAC-3′ (forward) (SEQ ID NO: 15) and 5′-AAG CTT GAG TGA ATA TGG TCT ATG GAT ATT-3′ (reverse) (SEQ ID NO: 16). The plasmid was transformed into pldα3-1 plants with the flower dipping method (Martinez-Trujillo et al., 2004). The transformants were selected from hygromycin plates and confirmed by PCR using primers TeasyAsc5: 5′-ATG GCG CGC CAT ATG GTC GAC CTG CAG-3′ (SEQ ID NO: 17) and TeasyAsc3: 5′-ATG GCG CGC CCG ACG TCG CAT GCT C-3′ (SEQ ID NO: 18). PCR or RT-PCR products were visualized by staining with ethidium bromide (EB) in 1% agarose gel after electrophoresis.

PLDα3 Plants Growth and Treatments. Plants of pldα3-1, OE, WT, and pldα3-1 complemented with PLDα3 (COM) were grown in soil in growth chambers under 12-h light/12-h dark photoperiods (120 μmol m⁻² s⁻¹) at 23/21° C. and 50% humidity. For salt stress experiments, three-week-old plants were treated with various concentrations of NaCl. Meanwhile, four-day-old seedlings of pldα3-1, OE, WT, and COM plants were transferred to MS (1×) agar plates containing 50 and 100 mM NaCl to test salt tolerance. For water stress experiments, three-week-old plants (before inflorescence formation) were not watered for several days until soil water content was 25-30% of soil water capacity (soil saturated with water). The water deficient condition was maintained by adding 50 ml of water to each pot (12×12×14 cm) every four days. Under this condition, the relative water content (RWC) of leaves was 50%, whereas RWC for well-watered plants was approximately 80%. For seed germination in response to osmotic stress, or hormone treatment, seeds were germinated in MS (1×) agar plates supplemented with NaCl, PEG, or ABA. To minimize experimental variation, plants of similar size of different genotypes were grown in the same pots or same plates for stress treatments.

Expression, purification, and PLDα3 activity assay. The PLDα3 gene was amplified from Arabidopsis genomic DNA using PLDα3 gene specific primers: 5′-CTC GAG ATG ACG GAC CAA TTG CTG CTT CAT CG-3′ (forward primer) (SEQ ID NO: 19), and 5′-ACG CCT AGA AGT AAG GAT GAT TGG AGG AAG A 3′ (reverse primer) (SEQ ID NO: 20), introducing cloning sites of XhoI/StuI. The PLDα3 sequence was fused with DNA encoding an HA-tag and cloned into a binary pKYLX71 vector. HA-tagged PLDα3 was expressed in Arabidopsis plants under the control of the 35S promoter. The C-terminally tagged PLDε3-HA protein was purified from plant proteins by immunoaffinity column chromatography using HA antibodies conjugated to agarose beads. The purified protein was used for activity assays with dipal, mitoylglycero-3-phospho-(methyl-³H)choline (³H-PC) as a substrate under different conditions previously defined for other PLDs (Pappan et al., Biochem Biophys. 353:131-140, 1997; Wang and Wang, Plant Physiol. 127:1102-1112, 2001; Qin and Wang, Plant Physiol. 128: 1057-1068, 2002). Briefly, PLDα1 activity was assayed in the presence of 25 mM Ca²⁺, 100 mM MES at pH 6, 0.5 mM SDS, and 2 mM PC. PLDβ and γ were assayed using 5 μM Ca²⁺, 80 mM KCl, 2 mM MgCl₂, 100 mM MES at pH 7, and 0.4 mM lipid vesicle composed of PC:PE:PIP₂ (0.2:3.5:0.3). The PLDδ reaction condition was 100 mM MES at pH 7, 2 mM MgCl₂, 80 mM KCl, 100 μM CaCl₂, 0.15 mM PC, and 0.6 mM oleate. PLDζ1 activity was measured in the presence of 100 mM Tris-HCl at pH 7, 80 mM KCl, and 0.4 mM lipid vesicles composed of PC:PE:PIP₂ (0.2:3.5:0.3) (Qin and Wang, 2002, supra). Hydrolysis of PC was quantified by measuring the release of [³H] choline by scintillation counting.

PLDα3 Real Time PCR. Real time PCR was performed as described by Li et al., Nat Biotechnol. 22: 427-433, 2006. Briefly, total RNA was extracted from leaves using a CTAB method. DNA was removed from RNA by digestion with RNase-free DNasel. RNA was used as a template for reverse transcription to synthesize cDNA using the iScript kit (Bio-Rad). Quantitative real time PCR was performed with a MyiQ sequence detection system (Bio-Rad) by monitoring SYBR green fluorescent labeling of double strand DNA synthesis. The efficiency of the cDNA synthesis was assessed by real-time PCR amplification of a control gene encoding UBQ10 (At4g05320) and the UBQ10 gene Cₜ value was 20±0.5. Only cDNA preparations that yielded similar Cₜ values for the control genes were used for determination of PLD gene expression. The level of PLD expression was normalized to that of UBQ10 by subtracting the Cₜ value of UBQ10 from the Cₜ value of PLD genes (Li et al., 2006, supra). Expression levels of genes were normalized by comparison to UBQ10 gene. The primers for different genes were as follows: PLDα3: 5′-ATG GTT AAT GCA ACG GCA GAC GAG-3′ (forward) (SEQ ID NO: 21) and 5′-CCC GGT AAA TCG TCA TTT CGA GGA-3′ (reverse) (SEQ ID NO: 22); RD29B: 5′-ACA ATC ACT TGG CAC CAC CGT T-3′ (forward) (SEQ ID NO: 23) and 5′-AAC TCA CTT CCA CCG GAA TCC GAA-3′ (reverse) (SEQ ID NO: 24); RAB18: 5′-GCA GTC GCA TTC GGT CGT TGT ATT-3′ (Forward) (SEQ ID NO: 25) and 5′-ACA ACA CAC ATC GCA GGA CGT ACA-3′ (reverse) (SEQ ID NO: 26); TOR: 5′-AGT TCG AAG GGC AAA GTA CGA CGA-3′ (forward) (SEQ ID NO: 27) and 5′-TAC GCA CGC TCA TAG CTC TCC AAA-3′ (reverse) (SEQ ID NO: 28); AGC2.1: 5′-AGA AAC GTC TCT TCC GCT TCA CCA-3′ (forward) (SEQ ID NO: 29) and 5′-ACC TGA AGA ATC TGA CAC GGC CAA-3′ (reverse) (SEQ ID NO: 30); FT: 5′-TCC CTG CTA CAA CTG GAA CAA CCT-3′ (forward) (SEQ ID NO: 31) and 5′-ACG ATG AAT TCC TGC AGT GGG ACT-3′ (reverse) (SEQ ID NO: 32); BFT: 5′-ATT CAA ACA GAG AGG GAG GCA AGC-3′ (forward) (SEQ ID NO: 33) and 5′-GCA GCA ACA GGT TGA GAA AGA CCA-3′ (reverse) (SEQ ID NO: 34); TSF: 5′-AAG ACA AAC GGT TTA TGC ACC GGG-3′ (forward) (SEQ ID NO: 35) and 5′-TTG AAG TAA GAG GCA GCC ACA GGA-3′ (reverse) (SEQ ID NO: 36); UBQ10: 5′-CAC ACT CCA CTT GGT CTT GCG T-3′ (forward) (SEQ ID NO: 37) and 5′-TGG TCT TTC CGG TGA GAG TCT TCA-3′ (reverse) (SEQ ID NO: 38). The PCR conditions were: 1 cycle of 95° C. for 1 minute, 40 cycles of 95° C. for 30 seconds for DNA melting, 55° C. for 30 seconds for DNA annealing, and 72° C. for 30 seconds for DNA extension, and 72° C. for 10 minutes for final extension of DNA.

PLDα3 Immunoblotting and Detection of Phosphorylated S6K. Total proteins were extracted from plants or seedlings grown in different conditions using buffer A (50 mM Tri-HCl, at pH 7.5, 10 mM KCl, 1 mM EDTA, 2 mM DTT, and 0.5 mM PMSF). After centrifugation at 6000 g for 10 min, the supernatant proteins were separated by 10% SDS-PAGE. After electrophoresis, proteins were transferred to a PVDF membrane. The membrane was blotted with anti-HA antibody (1:1000) overnight, followed by incubation with a second antibody (1:5000) conjugated with alkaline phosphatase. The protein bands were visualized by alkaline phosphatase reaction. For detecting phosphorylated S6K, proteins were transferred to nitrocellulose membranes and blotted with an anti-phospho-p70 S6K (Thr389) antibody (Cell Signaling TECHNOLOGY, Beverly, Mass.), followed by a secondary antibody conjugated with horseradish peroxidase (HRP). The rabbit polyclonal antibodies were raised against human p70 S6K and have been shown to react with plant S6K proteins (Reyes de la Cruz et al., 2004). The membranes were preblotted with TBS/T containing 5% BSA and then were incubated with the first antibody (1:1000) in TBS/T buffer. After gentle agitation at room temperature for 1 h, the membranes were washed with TBS/T four times. A polyclonal anti-rabbit IgG antibody conjugated with HRP (1:10000) was added and incubated for 1 h followed by three washes with TBS/T and three washes with PBS buffer. After incubation of LumiGLO substrate for 1 min, membranes were exposed to X-ray film.

PLDα3 Lipid Profiling and ABA Measurement. Lipid profiling was performed as described previously (Devaiah et al., Phytochemistry 67: 1907-1924, 2006). Briefly, leaves were detached and immediately immersed in 3 ml of 75° C. isopropanol with 0.01% butylated hydroxytoluene for 15 min, followed by the addition of 1.5 ml chloroform and 0.6 ml H₂O. After shaking for one hour, the extracting solvent was transferred to a clean tube. The leaves were re-extracted with chloroform:methanol (2:1) five times with agitation for 30 minutes each, and the extracts were combined and then washed with 1 M KCl, followed by another wash with H₂O. The solvent was evaporated with a stream of nitrogen. For each treatment, four leaf samples were extracted and analyzed separately. For ABA analysis, fresh leaves (100 mg) were ground in liquid nitrogen. 0.5 ml of 1-propanol:H₂O:HCl (2:1:0.002) was immediately added to the homogenate and mixed well. The homogenate was agitated at 4° C. for 10 min followed by addition of 1 ml dichloromethane and ABA internal standards. After vortexing and agitation at 4° C. for 10 min, the mixtures were centrifuged at 11,300×g for 1 min to separate the two phases. The lower phase was transferred to a 1.5 ml vial with a Teflon-lined screw cap. ABA was quantified by mass spectrometry as described by Pan et al. (2008, in press).

PLDα3 Relative Water Content, Ion Leakage, and Chlorophyll. Leaves were detached and fresh weight (FW) was measured followed by incubation in clean water overnight to obtain turgor weight (TW). Leaves were then dried at 80° C. for 48 hours to measure dry weight (DW). The relative water content (RWC) was obtained based on the equation: RWC (%)=(FW−DW)/(TW−DW)*100. To measure ion leakage, leaves were detached and rinsed with distilled H₂O, and then were immersed in 15 ml distilled H₂O in glass tubes. After degassing under vacuum for 30 min to remove air bubbles on the leaf surface, samples were incubated with gentle agitation for 3 h (Fan et al., Plant Cell 9: 2183-2196, 1997). Initial conductivity was measured with a conductivity meter and then the samples were boiled in a water bath for 20 min. Total conductivity was measured again after cooling to room temperature. Ion leakage was expressed as a percentage of the initial conductivity over total conductivity. For chlorophyll content measurement, chlorophyll was extracted from leaf discs placed in sealed vials with an appropriate volume of 100% methanol by shaking in the dark until the leaves became white. The chlorophyll content was obtained based on the absorbance of extracts at 650 and 665 nm (Crafts-Brandner et al., Plant Physiol. 75: 318-322, 1984).

Sequence Accession Numbers. A number of the sequence data can be found in the Arabidopsis Genome Initiative database under the following accession numbers: PLDε (AT1G55180.1), PLDα3 (At5g25370); RD29B (At5g52300); RAB18 (At5g66400); TOR (At1g50030); AGC2.1 (At3g25250); FT (At1g65480); BFT (At5g62040); TSF (At4g); and UBQ10 (At4g05320).

Results

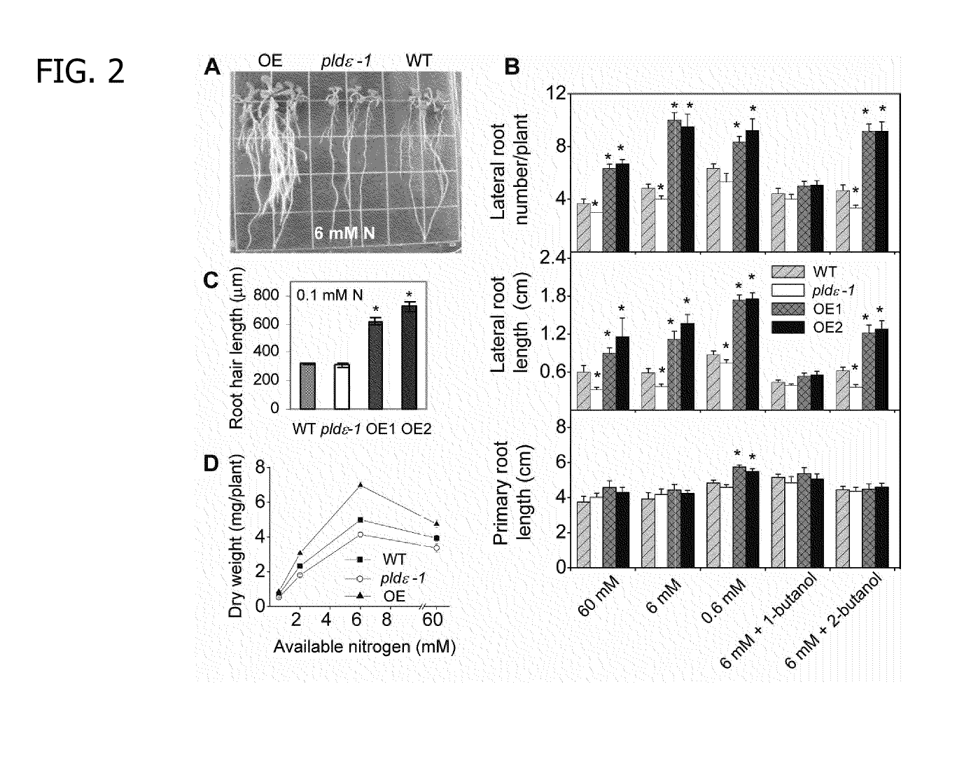

PLDε Promotes Root Growth and Nitrogen Use Efficiency. The difference in growth under two fertilizer levels prompted investigation of the role of PLDε in plant response to N levels with defined N composition and concentrations. Four-day-old seedlings of WT, KO, and two OE lines were transferred to MS agar plates containing 0.1, 0.6, 2, 6, and 60 mM nitrogen (NO₃⁻:NH₄⁻=2:1). In all N levels tested, OE plants grew more and longer, whereas KO had fewer and shorter, lateral roots than WT (FIG. 2A, B). At 6 mM N, the number and length of lateral roots of OE plants were two fold higher than those of WT and KO plants. The primary root length was not different among OE, WT, and KO at 6 and 60 mM N, but under severely N-limited conditions (0.6 mM), it was about 20% longer in OE than in WT and KO plants (FIG. 2B). When seeds were germinated directly in N-limited conditions (0.1, 0.6, and 2 mM), both primary and lateral roots of PLDε-KO were shorter than WT, whereas those of PLDε-OE seedlings were longer than WT (data not shown).

In addition, under N-limited conditions, root hairs in PLDε-OE plants were more than twice the length of WT and KO plants (FIG. 2C), whereas the root hair density was not different among the genotypes (supplemental FIG. 2).

Biomass measurements showed a substantial increase in dry matter in PLDε-OE, but a decrease in KO plants compared to WT (FIG. 2D). At all N levels tested, KO plants accumulated on average about 80% dry matter of WT, with a greater decrease occurring at lower (0.6 and 2 mM) than higher (6 and 60 mM) N levels. By comparison, N use efficiency, calculated as dry matter divided by N supplied, in OE plants was approximately 20, 30, and 40% higher than that of WT as N levels increased from 0.6, 2, to 6 mM, respectively. The trend of increase is similar in soil grown plants that had greater biomass increase in well-fertilized than poorly fertilized soil (FIG. 1F). The changes in biomass production are also consistent with the different effects of PLDε-OE and KO on root growth. These results show that the basal PLDε is needed for primary and lateral root growth, particularly under nitrogen-limited condition.

PLDε-derived PA Enhances Growth. PLDε hydrolyzes membrane lipids to generate PA and a head group. Arabidopsis seedlings were transferred to growth media containing 1-butanol or 2-butanol to investigate whether PLD-produced PA is involved in growth alteration. PLD uses 1-butanol, but not 2-butanol, as substrate to form phosphatidylalcohol at the expense of PA. Thus, 1-butanol treatment was expected to suppress PLD-mediated PA production without inhibiting PLD degradation of membrane lipids. 1-Butanol inhibited the number and length of lateral roots in all genotypes, but the magnitude of inhibition by 1-butanol was greatest on OE plants and smallest on KO plants (FIG. 2B). No significant difference in the number and length of lateral roots was observed among OE, WT, and KO plants after the 1-butanol treatment. 1-Butanol also inhibited biomass accumulation, and the greatest reduction occurred with PLDε-OE plants (data not shown). In contrast, the control treatment with 2-butanol exhibited no growth inhibitory effect at the concentration tested (FIG. 2B).

The level of PA in plants was measured to determine whether the PA production was altered by KO and OE of PLDε. The leaf PA content from soil-grown KO plants was approximately 50% lower, whereas in OE it was 15% higher, than that of WT (FIG. 3A). To measure PA changes in roots, seedlings were grown on plates with defined nitrogen levels.

The level of PA in KO roots was only 67% of WT, whereas OE was slightly higher than WT at 2 mM N (FIG. 3B). The levels of major membrane phospholipids, including PC, PG, MGDG, and DGDG, were similar among KO, OE, and WT roots. However, the PE level was higher in KO than WT, but lower in OE than WT roots. The inverse changes in PA and PE suggest that most PA is derived from PLDε hydrolysis of PE. These results show that PLDε is active in PA production, and also indicate that PLDε-produced PA is involved in growth promotion.

PLDε Is Associated with Membranes and Active under Broad Reaction Conditions. To demonstrate that PLDε encodes a functional PLD, PLDε was isolated from OE plants for biochemical analyses. PLDε was detected in the microsomal, but not in soluble fractions (FIG. 3C). By comparison, a majority of PLDα2 was found in soluble fractions. Most PLDε was associated with the plasma membrane, whereas more PLDα2 was associated with the intracellular membrane than the plasma membrane (FIG. 3C). PLDε was purified by immuno-affinity chromatography and assayed in the reaction conditions that were defined previously for PLDα1, β, δ, and ζ. PLDε was active at the PLDα1 reaction condition that included 50 mM Ca²⁺, SDS, and single-lipid class vesicle. However, none of the other previous characterized PLDβ, γ, δ, or ζ displayed activity under the PLDα1 condition. With micromolar Ca²⁺, PLDε required oleic acid for activity, a condition defined for PLDδ (FIG. 3D). PLDε also displayed some activity under PLDβ and γ conditions that were assayed in the presence of micromolar Ca²⁺, PIP2 and PE. As a control, PLDα2 was assayed under the same conditions and was active only under the PLDα1 reaction condition. PLDε hydrolyzed the common membrane phospholipids PC, PE, and PG, and had a low activity on PS, but no activity on PI or PIP2 when the enzyme was assayed with single class lipid vesicles (FIG. 3E).

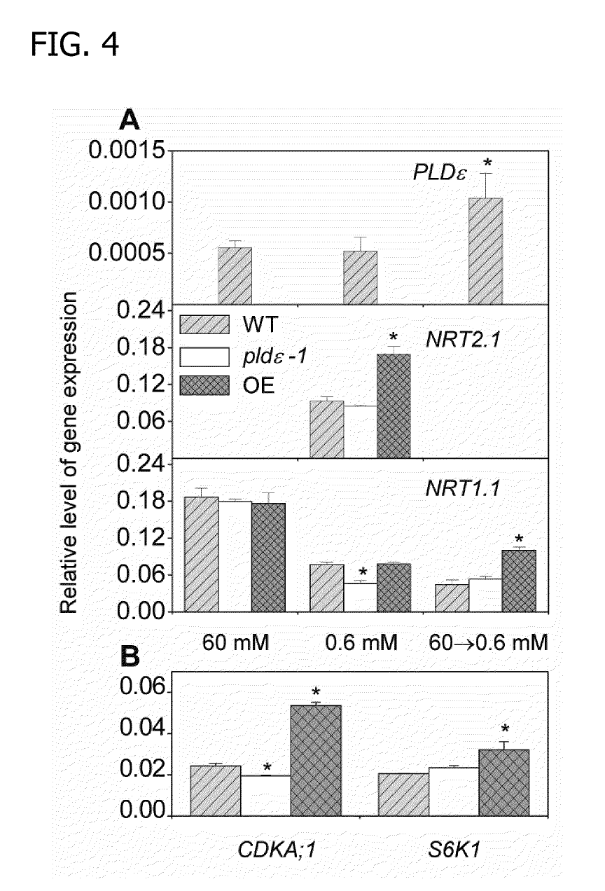

PLDε Alterations Change Nitrate Transporter Expression. To gain insights as to how PLDε enhances plant growth, the expression of two nitrate transporters, NRT1.1 and NRT2.1, which coordinate N absorption under different levels of N, was measured. NRT1.1 is regarded as a low-affinity and high-capacity nitrate transporter (Tsay, Y. F., Schroeder, J. I., Feldmann, K. A. & Crawford, N. M. (1993) Cell 72, 705-713; Huang, N. C., Chiang, C. S., Crawford, N. M. & Tsay, Y. F. (1996) Plant Cell 8, 2183-2191) whereas NRT2.1 is a high-affinity, low-capacity nitrate transporter (Cerezo, M., Tillard, P., Filleur, S., Muños, S., Daniel-Vedele, F. & Gojon, A. (2001) Plant Physiol. 127, 262-271). The mRNA level of NRT1.1 was high, whereas that of NRT2.1 was undetectable in all genotypes under an N-rich condition. When seedlings were transferred from 60 mM N to a N-limited condition (0.6 mM), the expression of the high-affinity NRT1.1 in OE was higher than that of WT and KO plants (FIG. 4A low panel). When seedlings were germinated and grown under N-limited condition, the level of NRT1.1 expression was lowest in KO whereas the level for NRT2.1 was highest in OE seedlings (FIG. 4A). The opposite expression levels of the nitrate transporters in OE and KO plants suggest that PLDε promotes nitrogen acquisition. The expression of PLDε itself was induced two-fold when seedlings were transferred from a N-rich (60 mM) to a N-limited (0.6 mM) condition (FIG. 4A, upper panel). This change was consistent with the results from GENEVESTIGATOR (www.genevestigator.ethz.ch), which also showed that PLDε was induced by N limitation.

PLDε Affects the Level of Ribosomal S6 Kinase. To identify the target of PLDε and PA in plant growth and N response, the effect of the alterations of PLDε on expression and protein levels of the 40S ribosomal S6 kinase (S6K) was determined. S6K is a functional homolog of animal p70 S6K that is a conserved key component of signaling pathways regulating cell and organismal size in animals (Wullschleger, S., Loewith, R. & Hall, M. (2006) Cell 124, 471-484). In addition, PLD1-derived PA in mammals has been shown to promote S6K activity and cell growth (Fang, Y., Vilella-Bach, M., Barchmann, R., Flanigan, A. & Chen, J. (2001) Science 294, 1942-1945; Fang, Y., Park, I. H., Wu, A. L., Du, G., Huang, P., Frohman, M. A., Walker, S. J., Brown, H. A. & Chen, J. (2003) Curr Biol. 13, 2037-2044). The expression level of S6K1 was higher in OE than WT and KO plants, but the difference was relatively small and no difference was detected between KO and WT plants (FIG. 4B). By comparison, the expression level of CDKA;1, which encodes an A-type cyclin-dependent kinase and regulates cell cycle, increased in OE and decreased in KO plants (FIG. 4B).

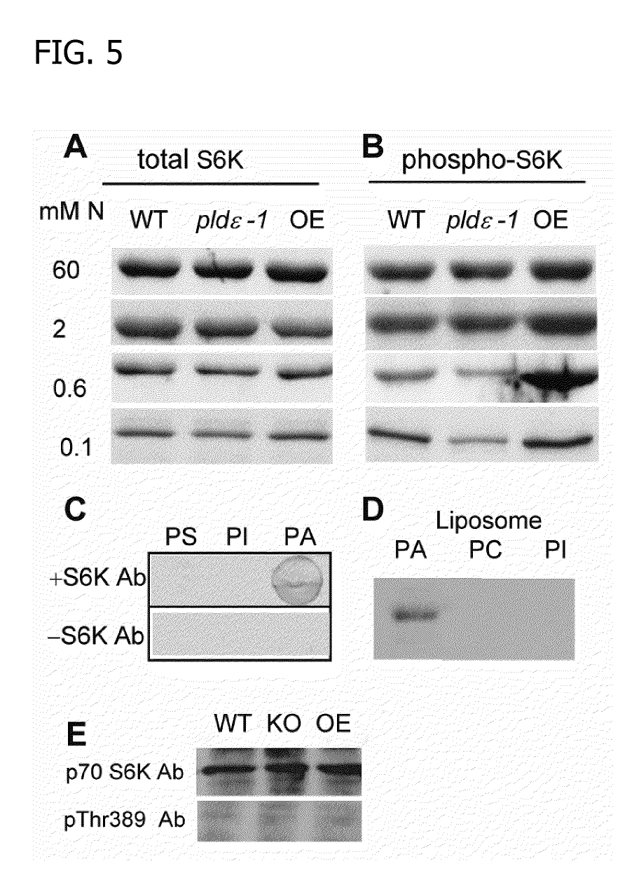

One mechanism that activates S6K is via phosphorylation. To detect the level of S6K protein and its phosphorylation, two antibodies were used, one against human p70 S6K and the other against phospho-p70 S6K (Thr389). The residue Thr389 is conserved between Arabidopsis and human S6Ks, and both antibodies have been shown to react with plant S6K (Reyes de la Cruz, H., Aguilar, R. & Sanchez de Jimenez, E. (2004) Biochemistry 43, 533-539). The level of total S6K protein was affected by the N availability; its level decreased under N-limited condition (0.1 and 0.6 mM) (FIG. 5A). The total protein level of S6K was similar among WT, KO, and OE plants, but the level of phosphorylated S6K in PLDε-KO plants was lower than in WT and OE plants under severe N-limited conditions (0.1 and 0.6 mM) (FIG. 5B). In contrast, the level of phosphorylated S6K in OE plants was higher than WT and KO plants.

In addition, PA interaction with S6K was analyzed. S6K bound to PA, but not to other tested phospholipids immobilized on a nitrocellulose filter (FIG. 5C). To verify the binding, liposomes composed of PA:PC, PC only, or other lipids were incubated with proteins isolated from Arabidopsis leaves or seedlings, followed by detection of S6K. Total S6K was associated with PA:PC, but not PC only or other lipid liposomes (FIG. 5D). However, no S6K was detected by the phospho-p70 S6K antibody (FIG. 5E), suggesting that PA binds to non-phosphorylated, but not to phosphorylated S6K.

Expression, Reaction Conditions, and Substrate Usage of PLDα3. Arabidopsis expressed sequence tag (EST) database searches revealed a number of EST clones corresponding to PLDα1, but none for PLDα3, indicating that the level of PLDα3 expression is much lower than that of PLDα1. This is supported by quantitative real-time PCR data showing that the average level of PLDα3 expression in buds, flowers, siliques, stems, old leaves, and roots is approximately 1000-fold lower than that of PLDα1. Otherwise, the expression patterns in the different organs were similar between the two PLD genes (FIG. 7A). The results are consistent with the expression levels and patterns of PLDα1 and PLDα3 expression in different organs as determined by querying GENEVESTIGATOR (www.genevestigator.ethz.ch).

To determine whether PLDα3 encodes a functional PLD, the gene was tagged at the C-terminus with HA and expressed in Arabidopsis (FIG. 7B). HA tagged PLDα3 was purified and PLD activity was assayed at Ca²⁺ concentrations and conditions previously defined for PLDα1, β, δ, and ζ (Pappan et al., 1998, supra; Wang and Wang, 2001, supra; Qin and Wang, 2002, supra). PLDα3 was active under PLDα1 reaction conditions that included 50 mM Ca²⁺, SDS, and single-lipid-class vesicle (FIG. 7C). PLDα3 was inactive under PLDβ, γ, or ζ conditions, which included phosphatidylinositol 4,5-bisphosphate (PIP₂), phosphatidylethanolamine (PE), and micromolar (μM) or no Ca²⁺ in the reaction mixtures. PLDα3 displayed low activity under PLDδ conditions that included μM Ca²⁺ and oleic acid (FIG. 7C). PLDα3 hydrolyzed the common membrane phospholipids, phosphatidylcholine (PC), PE, phosphatidylglycerol (PG), and phosphatidylserine (PS), having the highest activity toward PC and the lowest toward PS (FIG. 7D). PLDα3 had no activity toward phosphatidylinositol (PI) or PIP₂ when assayed with single class lipid vesicles.

Manipulations of PLDα3 Alter Plant Sensitivity to Salinity. A T-DNA insertion mutant of PLDα3 was isolated to evaluate cellular functions. The PLDα3-KO, designated as pldα3-1, has a T-DNA insertion in the second exon, located 739 bp from the start codon (FIG. 8A). The homozygosity of the mutant was confirmed by PCR using PLDα3-specific primers and a T-DNA left border primer (FIG. 8B). The mutation resulted in loss of the expression of PLDα3 as indicated by the absence of a detectable PLDα3 transcript by RT-PCR. Thus, pldα3-1 is a knockout mutant (FIG. 8C). The mutant allele co-segregated with kanamycin resistance and susceptibility in a 3:1 ratio, suggesting a single T-DNA insertion in the genome. In addition, more than 30 independent transgenic Arabidopsis lines overexpressing HA-tagged PLDα3 (PLDα3-OE) under the control of the cauliflower mosaic virus 35S promoter were generated, and expression of PLDα3-HA in the plants was confirmed by immunoblotting using HA-antibodies (FIG. 7B). A number of independent lines of OE plants were tested for their stress response, and their physiological effects were correlated with the level of overexpression. For further characterization, two or three representative independent transgenic lines were used in each experiment.

Wild-type (WT), OE, and pldα3-1 plants displayed no significant morphological alterations under control growth conditions. No apparent differences in growth and development were observed when seeds of these plants were germinated under nitrogen or phosphorus deficiency, water lodging, or in response to the growth regulators 1-aminocyclopropane-1-carboxylic acid, indole acetic acid, or cytokinin. However, pldα3-1 was more sensitive to salt stress than the WT was, whereas PLDα3-OE was less sensitive. In the absence of NaCl, nearly 100% of seeds of all genotypes germinated within 2 days (FIG. 8D). In the presence of 150 mM NaCl, the germination of pldα3-1 seeds was delayed, whereas that of PLDα3-OE was enhanced compared to WT in the early stage of germination (FIG. 8E). The seedling size and root length of PLDα3-OE were greater than WT, whereas those of pldα3-1 were smaller (FIG. 8G). When NaCl was increased to 200 mM, the germination of pldα3-1 was much slower, whereas that of PLDα3-OE was faster than that of WT (FIG. 8F). Introducing native PLDα3 into the pldα3-1 mutant (COM) restored the mutant phenotype to WT (FIG. 8D-F), confirming that the changes observed in the insertion mutant were caused by the loss of PLDα3.

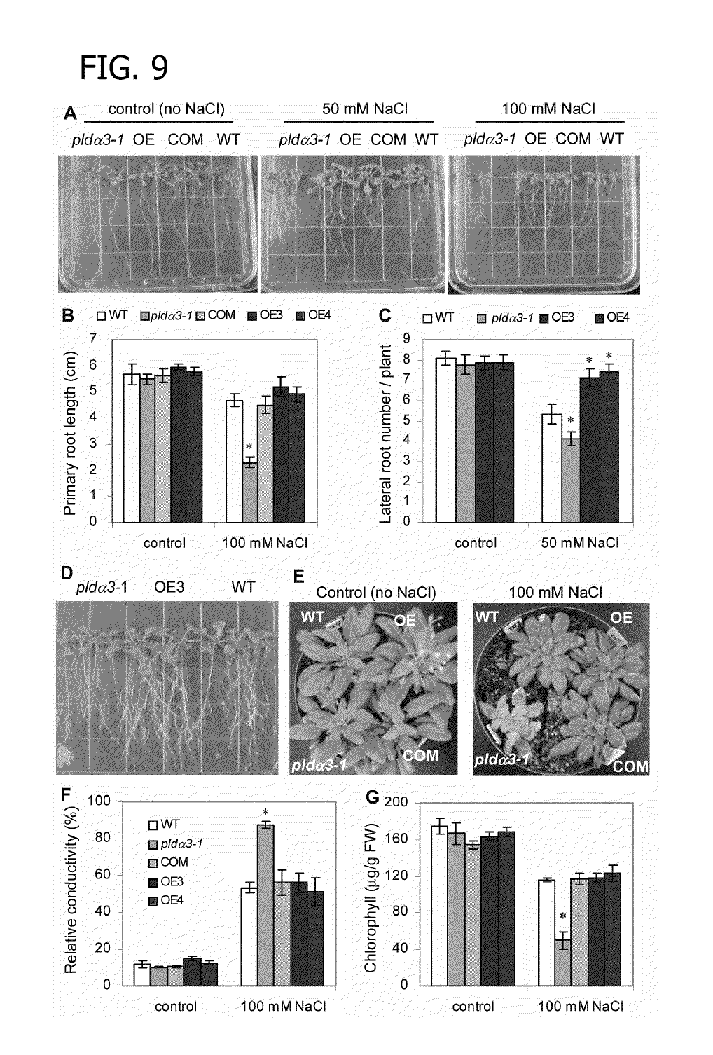

To further characterize the salt stress response, 4-day-old seedlings germinated under non-salt-stress conditions were transferred to MS agar plates containing 50 or 100 mM NaCl. Primary root growth was inhibited in pldα3-1 plants, and the length was about 50% that of WT plants (FIGS. 9A and B). PLDα3-altered plants also differed from WT in the number of lateral roots (FIG. 9C). One week after transfer to MS containing 50 mM NaCl, pldα3-1 seedlings had significantly fewer lateral roots per plant than PLDα3-OE or WT plants, and PLDα3-OE plants had significantly more lateral roots per plant than the WT (FIG. 9C). PLDα3-OE and WT plants had similar primary root lengths at the early stages of salt stress (FIG. 9B), but PLDα3-OE rosettes grew better than those of WT rosettes under prolonged salt stress (FIG. 9D). The pldα3-1 phenotype was restored to WT after genetic complementation with PLDα3 (FIGS. 9A and B).

To determine whether the altered salt stress response also occurred in plants grown in soil, 3-week-old plants were subjected to salt stress by irrigation with 100 mM NaCl. To minimize other effects, such as plant size and soil water content, during the salt treatment, PLDα3-altered plants were grown in the same pots with WT plants. pldα3-1 plants were more susceptible to salt stress than PLDα3-OE or WT plants. After 2 or 3 weeks of salt stress, pldα3-1 plants became yellow and eventually died, whereas WT and OE plants survived and grew to maturation (FIG. 9E). The rate of ion leakage, an indicator of membrane integrity, in pldα3-1 was much higher than in WT and OE plants (FIG. 9F). Chlorophyll content was also significantly lower in pldα3-1 than in WT plants (FIG. 9G).

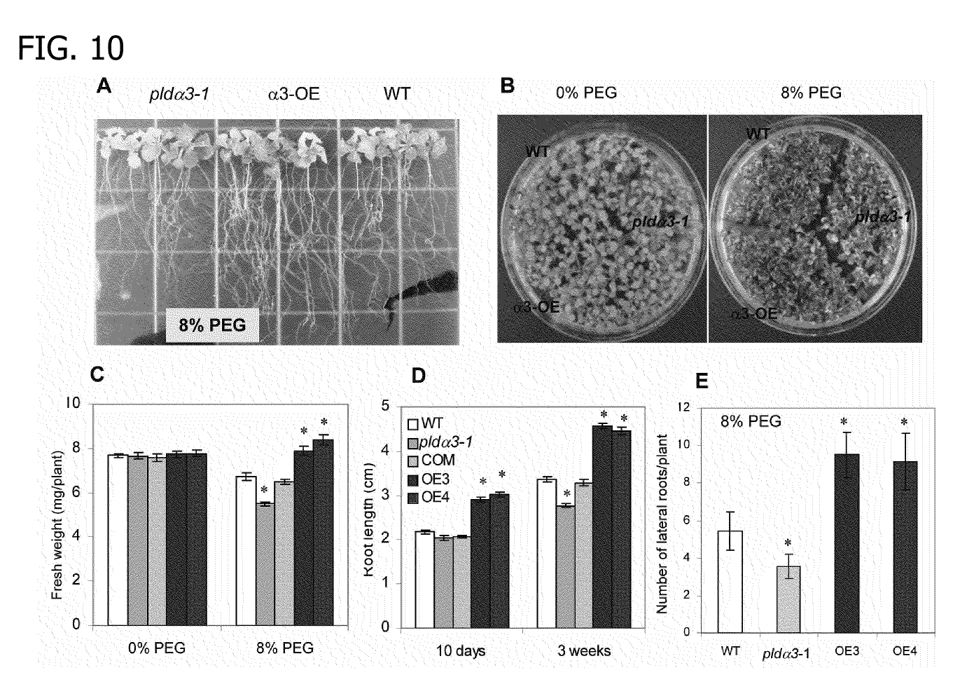

Alterations in PLDα3 Expression Change Plant Development under Water Deficits. To determine if the alteration was specific to salinity, pldα3-1, OE, and WT seedlings were tested for response to other hyperosmotic stresses. In the presence of 8% polyethylene glycol (PEG), the growth of pldα3-1 seedlings was inhibited whereas the OE seedlings grew better than WT (FIGS. 10A and B). Compared to WT seedlings, pldα3-1 seedlings had about 80% of the biomass accumulation and 20% shorter primary roots, whereas PLDα3-OE seedlings accumulated 25% more biomass and had longer primary roots and more lateral roots (FIG. 10C, D, E). These results indicate that ablation of PLDα3 decreases plant response to hyperosmotic stress, in addition to salt stress specifically.

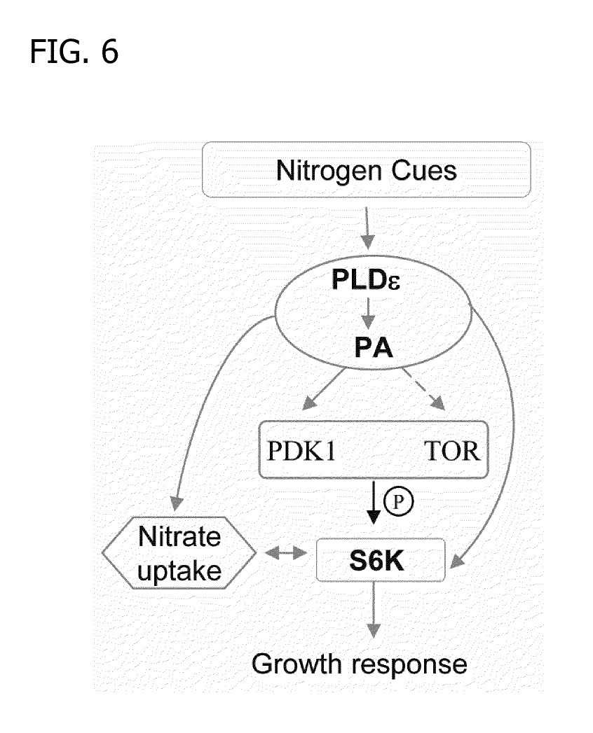

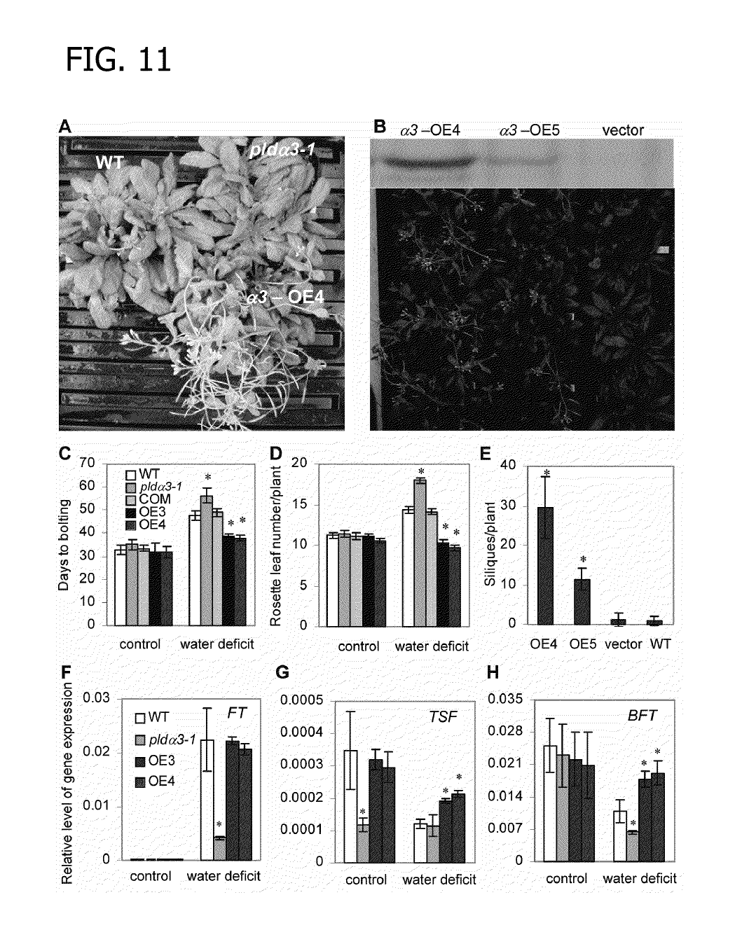

The effect of PLDα3 KO and OE was investigated in plants grown in soil with limited water supply. Water deficits were imposed on WT, pldα3-1, and OE plants at approximately 25-30% of soil water capacity (soil saturated with water). Under water deficit, the relative water content of the leaves was about 60% that of well-watered plants. Plants continued growing, but growth was slower than for plants grown under well-watered conditions. When water deficiency was chronic, PLDα3-OE plants flowered earlier and pldα3-1 plants flowered later than WT (FIGS. 11A, C, and D). On average, OE plants bolted and flowered 9 days earlier than did WT, but pldα3-1 flowered 6 days later than did WT. At the time of flowering, OE plants had 4 and 8 fewer rosette leaves than WT and pldα3-1 plants, respectively (FIG. 11D). The flowering time was also affected by the level of PLDα3 protein; the OE line with a higher level of PLDα3 flowered earlier than did plants with a lower level of PLDα3 (FIGS. 6B and 11B). The OE plants also had more siliques than WT and plants containing the empty vector (FIG. 11E). However, under well-watered growth conditions, WT, pldα3-1, and OE plants displayed no differences in flowering time or in number of rosette leaves or siliques.

The FLOWERING LOCUS T (FT) gene is a key integrator of signals that influence Arabidopsis flowering time (Corbesier et al., Science 316: 1030-1033, 2007; Mathieu et al., Curr Biol. 17: 1055-1060, 2007). Increases in the expression of FT promote flowering. Thus, we measured the expression patterns of FT and its paralogues, BROTHER of FT and TFL1 (BFT) and TWIN SISTER OF FT (TSF), by real time PCR. Under well-watered conditions, the expression levels of FT and BFT were not different among three week-old PLDα3-altered and WT plants, but levels of TSF were lower in pldα3-1 than in WT plants. Under water deficit conditions, the FT expression level was lower in pldα3-1 plants, whereas the expression levels of BFT and TSF were higher in OE than in WT and pldα3-1 plants at the inflorescence stage (FIGS. 11F to 11H). The trend of changes in expression of flowering timing markers is consistent with the different flowering times resulting from PLDα3 alterations.

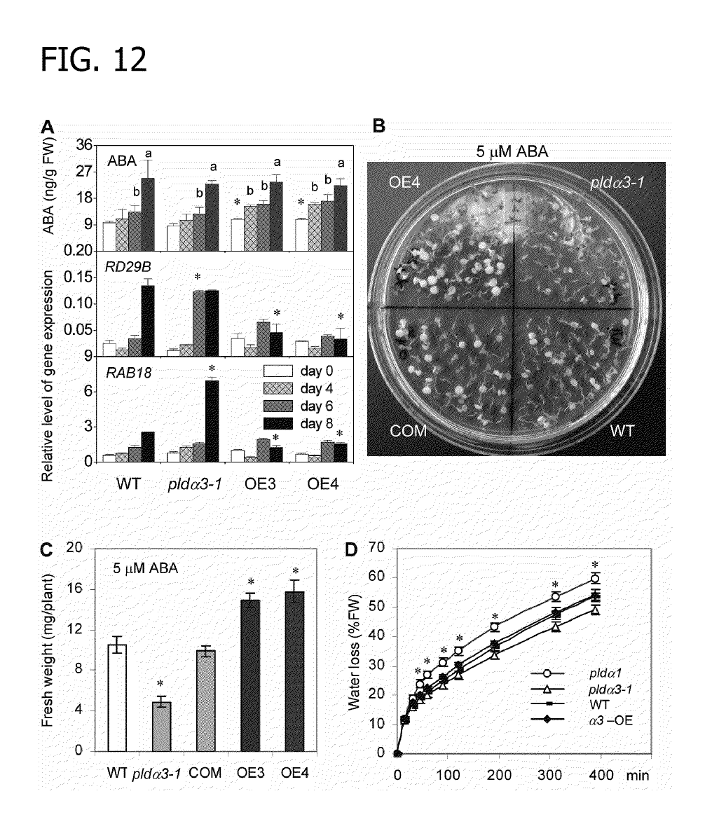

Changes in ABA Content and ABA Response under Osmotic Stress. The transition from vegetative to reproductive development is controlled by multiple environmental and endogenous factors. The hormone abscisic acid (ABA) regulates stress response, flowering, seed germination, and development. ABA is induced by drought stress and inhibits plant flowering (Bezerra et al., J Exp Bot. 55: 2331-2341, 2004; Razem et al., Nature 439: 290-294, 2006). To investigate if alterations of PLDα3 changed ABA level and ABA response, ABA content was measured in pldα3-1, OE, and WT plants under control and drought conditions (FIG. 12A). Under control growth conditions, the ABA content of OE plants was about 5% higher than WT, whereas the ABA content of pldα3-1 tended to be lower than that of WT, but the difference was not significant. When water was withheld, increases in ABA occurred in all three genotypes. However, when compared to day 0 of the same genotype, the significant increase occurred two days earlier in OE plants than in pldα3-1 and WT plants (FIG. 12A, upper panel). At eight days without watering, all genotypes had similar levels of ABA. The results indicate that altered expression of PLDα3 has a small, yet significant effect on the basal level of ABA, and that plants with ablation or elevation of PLDα3 are still capable of the drought-induced accumulation of ABA.

The expression of the ABA- and osmotic stress-responsive genes RAB18 and RD29B was monitored by quantitative real-time PCR. RAB18 or RD29B, the dessication-responsive gene that contain at least one cis-acting ABA-responsive element, has been widely used as a reporter for hyperosomotic stress and ABA response. The trend of basal levels of RD29B expression was similar to that of ABA levels among WT, pldα3-1, and OE plants under control growth conditions. However, RD29B expression in pldα3-1 increased greatly in day 6 without water, two days sooner than the expression increased in WT (FIG. 12A, middle panel). In OE plants, increases in RD29B expression also occurred, but the magnitude was much smaller than that of WT and pldα3-1 plants, even after eight days without water. Likewise, the expression of RAB18, another ABA-inducible gene also exhibited an earlier and larger increase in pldα3-1 than in WT plants, whereas that of OE plants was less induced by water deficits (FIG. 12A, bottom panel).

When seedlings were grown on MS media supplemented with ABA, the growth of pldα3-1 seedlings was more inhibited than the WT, whereas that of OE seedlings was less inhibited (FIG. 12B). The biomass accumulation of pldα3-1 was only 46% of WT, whereas that of OE was 145% of WT after 30 days of growth on MS containing 5 μM ABA (FIG. 12C). Without ABA, all three genotypes accumulated similar amounts of biomass (FIG. 10C). PLDα1 has been shown to be involved in the promotion of stomatal closure by ABA (Zhang et al., Proc Natl Acad Sci USA 101: 9508-9513, 2004; Mishra et al., Science 312: 264-266, 2006). KO of PLDα1 impeded stomatal closure and increased leaf water loss, but the water loss from detached leaves was not significantly different among PLDα3-KO, -OE, and WT plants (FIG. 12D).

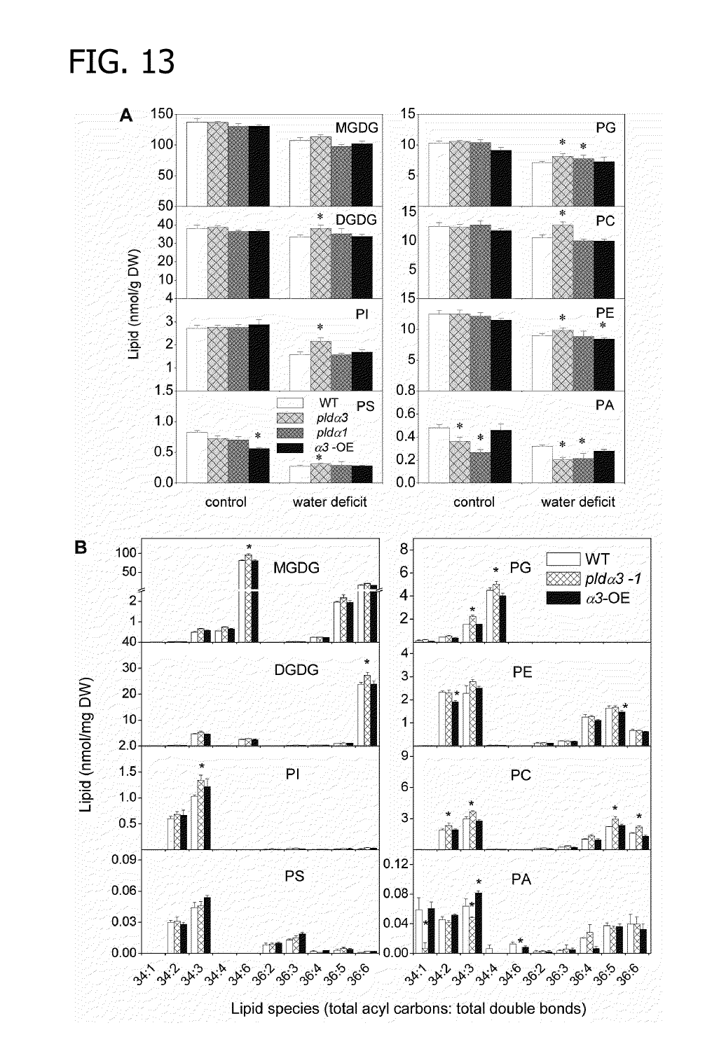

Effect of PLDα3 on PA Content and Lipid Composition. PLDα3 hydrolyzed various membrane phospholipids in vitro to produce PA (FIG. 7D). To determine the effect of PLDα3 on lipid composition, we quantitatively profiled glycerophospholipids and galactolipids of WT, pldα3-1, and OE plants. Under control growth conditions, the levels of PC, PE, PG, PS, monogalactosyldiacylglycerol (MGDG), and digalactosyldiacylglycerol (DGDG) were similar in pldα3-1 and WT plants. PA content in pldα3-1 was about 80% that of WT plants (FIG. 13A), indicating that PLDα3 contributed to the production of basal PA.

Water deficit induced a substantial loss of phospholipids and galactolipids (FIG. 13A). OE and WT plants underwent similar declines in all measured lipids, except in PE, which was significantly lower in OE than in WT plants. Compared to WT plants, pldα3-1 plants have higher levels of nearly all lipids, except for PA, which was approximately 60% that of the WT level (FIG. 13A). By comparison, under the same water stress condition, the effect of PLDα1-KO on lipid change was smaller than that of PLDα3-KO. The level of PG was higher and that of PA was lower in PLDα1-KO than WT plants (FIG. 13A).

The difference in PA among pldα3-1, OE, and WT was due primarily to differences in levels of 34-carbon PA species, which contain 18- and 16-carbon fatty acids (Devaiah et al., 2006) (FIG. 13B). In terms of potential substrate lipids, pldα3-1 had higher levels of both 34- and 36-carbon PCs, as well as higher levels of PG and PI although PI was not a substrate in vitro (FIG. 7D). 34:6 MGDG and 36:6 DGDG were also higher in pldα3-1, and 34:6 PA, which is likely to be formed by hydrolysis of 34:6 MGDG, was lower in pldα3-1 plants. The results indicate that PLDα3 was involved in drought-induced loss of glycerol polar lipids and changes in membrane lipid composition.

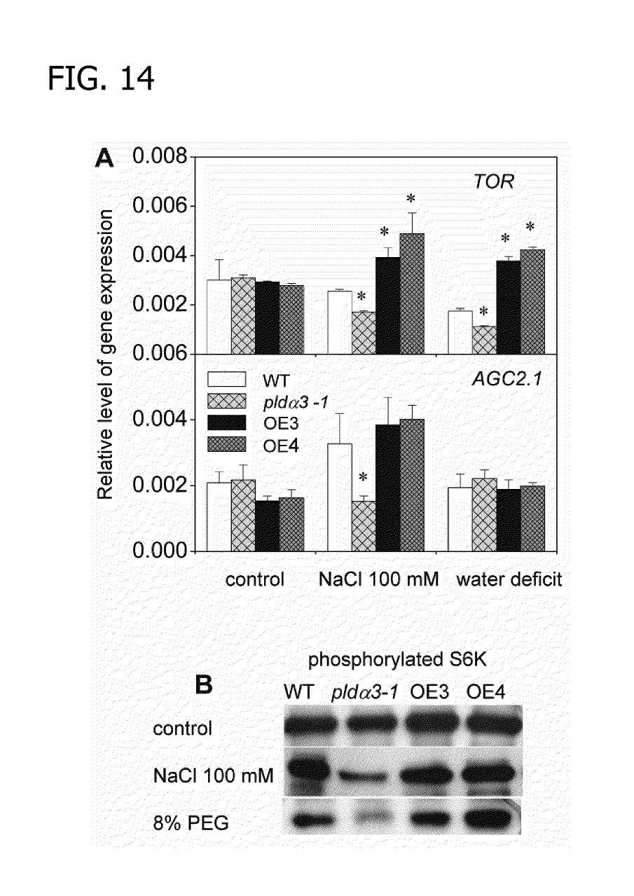

Changes in the Levels of TOR and AGC2.1 Expression. PLD-derived PA has been shown to activate mammalian target of rapamycin (mTOR) signaling that regulates protein synthesis, cell growth, and stress responses (Fang et al., 2000). TOR plays a role in cell growth and embryonic development in Arabidopsis, as well as in hyperosmotic stress (Menand et al., 2002; Mahfouz et al., Plant Cell 18: 477-490, 2006). The present results showed that alteration of PLDα3 changed PA level, osmotic tolerance, growth and development under salt and water deficit stresses. These observations led to testing of whether alterations of PLDα3 affected the TOR signaling pathway in the hyperosmotic response. The transcript level of TOR in PLDα3-altered plants was assessed under both salt stress and water deficiency conditions by real time PCR. The level of TOR expression was lower in pldα3-1 plants and higher in OE plants than in WT plants under both conditions (FIG. 14A). The expression of AGC2.1 kinase was monitored, whose activity was shown to be regulated by PA to promote root hair growth in Arabidopsis (Anthony et al., 2004). The transcript level of AGC2.1 kinase was significantly lower in pldα3-1 than in WT and OE plants under salt stress, but there was no difference in AGC2.1 expression between PLDα3-altered and WT plants under water-deficient conditions (FIG. 14A). These results suggested that alterations of PLDα3 affected the expression of AGC2.1 and TOR differently.

TOR regulates cellular activities by phosphorylation of downstream targets, such as ribosomal S6 kinase (S6K) that phosphorylates ribosomal proteins to promote translation. Data from GENEVESTIGATOR (www.genevestigator.ethz.ch) showed that S6K was induced by salt stress and it was further implicated in the hyperosmotic stress response in Arabidopsis (Mahfouz et al., 2006). To investigate whether the level of phosphorylated S6K was changed in PLDα3-altered plants, the proteins extracted from KO, OE, and WT plants were immunoblotted with an anti-phospho-p70 S6K antibody. Under control growth conditions, the level of phosphorylated S6K was not significantly different among KO, OE and WT plants. However, under salt and water deficit conditions, the level of phosphorylated S6K is lower in pldα3-1 plants than in WT (FIG. 14B). OE and WT plants had similar levels of phosphorylated S6K under the 100 mM NaCl condition, and OE had a higher level than WT under the water deficit condition (8% PEG) (FIG. 14B). Thus, the level of phosphorylated S6K was correlated with hyperosmotic tolerance.

1. A transgenic plant with altered phospholipase D epsilon (PLDε) expression relative to the corresponding wild-type plant.

2. The transgenic plant of claim 1, wherein phospholipase D epsilon (PLDε) is overexpressed.

3. The transgenic plant of claim 1, wherein phospholipase D epsilon (PLDε) is underexpressed.

4. The transgenic plant of claim 2, wherein the phospholipase D epsilon (PLDε) nucleic acid sequence comprises SEQ ID NO: 1.

5. (canceled)

6. The transgenic plant of claim 1, wherein the transgenic plant is a monocotyledonous plant or a dicotyledonous plant.

7. (canceled)

8. A transgenic seed with altered phospholipase D epsilon (PLDε) expression relative to the corresponding wild-type seed.

9. The transgenic seed of claim 8, wherein phospholipase D epsilon (PLDε) is overexpressed.

10. The transgenic seed of claim 8, wherein phospholipase D epsilon (PLDε) is underexpressed.

11. The transgenic seed of claim 9, wherein the phospholipase D epsilon (PLDε) nucleic acid sequence comprises SEQ ID NO: 1.

12. (canceled)

13. A method of producing a transgenic plant of claim 2, said method comprising:
(a) introducing an expression construct that comprises a polynucleotide encoding a phospholipase D epsilon (PLDε) polypeptide operably linked to a promoter which is capable of overexpressing the polypeptide into a plant cell to produce a transformed plant cell; and
(b) producing a transgenic plant from the transformed plant cell.

14. The method of claim 13, wherein the polynucleotide is operably linked to a promoter selected from the group consisting of a tissue-specific promoter, an inducible promoter and a constitutive promoter.

15. The method of claim 13, wherein the plant cell is a monocotyledonous plant cell or a dicotyledonous plant cell.

16. The method of claim 13, wherein the phospholipase D epsilon (PLDε) nucleic acid sequence comprises SEQ ID NO: 1.

17. (canceled)

18. A method for increasing a plant's ability to capture and utilize nitrogen, for increasing yield of a plant compared to the yield of a corresponding wild type plant, or for increasing a plant's biomass production, the method comprising overexpressing a phospholipase D epsilon (PLDε) in the plant.

19. The method of claim 18, wherein overexpressing the phospholipase D epsilon (PLDε) in the plant comprises introducing into the plant an expression construct that comprises a polynucleotide encoding a phospholipase D epsilon (PLDε) polypeptide operably linked to a promoter which is capable of overexpressing the polypeptide.

20. The method of claim 19, wherein the phospholipase D epsilon (PLDε) nucleic acid sequence comprises SEQ ID NO: 1.

21. (canceled)

22. The method of claim 18, wherein increasing the plant's biomass is selected from the group consisting of increasing seed number, increasing seed size, increasing leaf size, increasing cell number, increasing root size, and a combination thereof.

23. (canceled)

24. The method according to claim 18, wherein the increased yield comprises increased seed yield and the increased seed yield is selected from the group consisting of an increased number of filled seeds, increased total seed weight, and a combination thereof.

25. A method for increasing a plant's ability to grow under hyperosmotic stress conditions compared to a corresponding wild-type plant, the method comprising overexpressing a phospholipase D epsilon (PLDε) in the plant.

26. A transgenic plant which overexpresses phospholipase D alpha3 (PLDα3) relative to the corresponding wild-type plant.

27-46. (canceled)
